(19) United States
(12) Patent Application Publication
(10) Pub. No.: US 2023/0277583 A1
(43) Pub. Date: Sep. 7, 2023

(54) ENTERAL CARBON MONOXIDE FOR THE PREVENTION AND TREATMENT OF OCULAR DISEASES

(71) Applicants: Augusta University Research Institute, Inc., Augusta, GA (US); Hillhurst Biopharmaceuticals, Inc., Montrose, CA (US)

(72) Inventors: Pamela Martin, Evans, GA (US); Ravirajsinh Jadeja, Evans, GA (US); Andrew Gomperts, San Diego, CA (US)

(21) Appl. No.: 18/179,069

(22) Filed: Mar. 6, 2023

Related U.S. Application Data
(60) Provisional application No. 63/316,868, filed on Mar. 4, 2022.

(51) Int. Cl. A61K 33/00; A61P 27/02; A61P 3/10

(52) U.S. Cl. CPC A61K 33/00; A61P 27/02; A61P 3/10

(57) ABSTRACT

Enteral formulations containing including therapeutically effective amount carbon monoxide, and methods of use thereof, for treatment of ocular diseases and disorders are provided. The formulations can be administered in a subject in need thereof in an effective amount to reduce or prevent an ocular disease in the subject, and thus reduce one or more symptoms of disease, disorder, or illness associated with the disease. In some embodiments, the formulation is administered orally.

### CROSS REFERENCE TO RELATED APPLICATIONS

This application claims benefit of and priority to U.S. Provisional Application No. 63/316,868 filed Mar. 4, 2022, which is specifically incorporated by reference herein in its entirety.

### STATEMENT REGARDING FEDERALLY SPONSORED RESEARCH

This invention was made with government support under EY033264 awarded by The National Institute of Health. The government has certain rights in the invention.

### FIELD OF THE INVENTION

The field of the invention generally relates to ocular diseases and disorders and compositions and methods of treatment thereof.

### BACKGROUND OF THE INVENTION

Diseases, disorders, injuries of the eye, including the ocular surface, cornea, and other structures, negatively impact vision and quality of life, and are often not adequately treated due to the need for more effective treatment options. Patients may experience a range of problems and sequelae including loss of visual acuity due to cornea, retina and other structures, blurred vision, photophobia, pain, discomfort, congestion, itching and blindness. Chronic ocular diseases and conditions represent a significant medical burden. For example, Diabetic retinopathy (DR) is a neurovascular complication of diabetes mellitus and the leading cause of blindness in adults worldwide (Ogurtsova, et al., Diabetes Res Clin Pract, 128: p. 40-50 (2017), WHO. Global Initiative for the Elimination of Avoidable Blindness: action plan 2006-2011, who.int/blindness/Vision2020_report.pdf. (2007)), affecting approximately 4.2 million diabetes patients in the US, of which 655,000 have vision-threatening DR (US CDC, cdc.gov>visionhealth>pdf>factsheet (2020)). Although DR therapies, including photocoagulation and intravitreal anti-VEGF drugs, are available, these therapies have substantial limitations in that they target advanced DR and are only effective in a subset of patients, highly invasive, and associated with risk of infection or fibrosis (American Diabetes Association, Diabetes Care, 41(Suppl 1): 5105-5118. PMID:29222381 (2018), Kansora & Goldhardt, Curr Ophthalmol Rep., 7(1):45-50. PMID: 31595210 (2019), Virgili, et al., Cochrane Database Syst Rev., 6:7419 (2017)).

Retinal ischemia is a major cause of vision loss in common retinal disease conditions including DR (Osborne, et al., Prog Retin Eye Res, 23(1): 91-147 (2004)). Previous studies show that oxidative stress and inflammatory processes contribute significantly to pathology development in ischemic retinal diseases, specifically DR (River, et al., Oxid Med Cell Longev., volume 2017, Article ID: 3940241, PMID: 29410732 (2017)). A number of anti-inflammatory/anti-oxidative therapeutics are being tested as treatments for DR (Sadiq, et al., Int J Retina Vitreous., 6:(29); PMID: 32670612. (2020)).

Heme oxygenase-1 (HO-1) is a pivotal enzyme for the cellular response against oxidative stress stimuli (Araujo, et al., Front Pharmacol, 3(Article 119) (2012)). In the retina, HO-1 is expressed by pigmented epithelial cells, photoreceptors, ganglion cells, glial cells, and endothelial cells (Bailey, et al., Ophthalmol Vis Sci, 45(2):675-84 (2004), Cukiernik, et al., Curr Eye Res, 27(5):301-8 (2003)), all of which are cell types impacted significantly in the retina of diabetic patients. Further, HO-1 expression is increased under conditions of retinal stress (Alizadeh, et al., Invest Ophthalmol Vis Sci., 42(11):2706-13 (2001), Ulyanova, et al., Invest Ophthalmol Vis Sci., 42(6):1370-4 (2001)). Moreover, the up-regulation of HO-1 has been shown to be protective in a DR model (Fan, et al., Invest Ophthalmol Vis Sci., 53(10): 6541-56. PMID: 22661484 (2012)). The precise mechanism (s) by which HO-1 confers protection against cellular stress and tissue injury has not been defined but accepted evidence points to the generation of carbon monoxide (CO). The use of CO to achieve cellular protection has become an accepted therapeutic approach (Yang, et al., Med Res Rev., 40(4):1147-1177 (2019), Ryter, Arch Biochem Biophys, 678: Article 108186. PMID: 31704095 (2019), Cheng & Rong, Curr Pharm Des, 2017. 23(26): p. 3884-3898 (2017)). Previous studies have documented the protective effects of CO in rodent models of ischemia-reperfusion (I/R) injury, optic nerve crush, and uveitis (Stifter, et al., PLoS One, 12(11): p. e0188444 (2017), Wang, et al., Mol Med Rep, 17(1): p. 1297-1304 (2018), Ulbrich, et al., J Neuroinflammation, 14(1): p. 130 (2017), Ulbrich, et al., PLoS One, 11(10): p. e0165182 (2016), Ulbrich, et al., Graefes Arch Clin Exp Ophthalmol, 254(10): p. 1967-1976 (2016), Chen, et al., Biochem Biophys Res Commun, 469(4): p. 809-15 (2016), Fagone, et al., Clin Immunol, 157(2): p. 198-204 (2015), Schallner, et al., PLoS One, 7(9): p. e46479 (2012), Biermann, et al., Invest Ophthalmol Vis Sci, 51(7): p. 3784-91 (2010)).

Although CO is a molecule with known toxicity at high doses, previous studies have shown that low dose CO functions as a regulatory molecule providing therapeutic effects in many cellular and biological processes akin to nitric oxide (Motterlini & Otterbein, Nat Rev Drug Discov., 9:728-743 (2010), Morita, et al., Proc Natl Acad Sci U SA., 92(5):1475-1479. PMCID: PMC42542 (1995), Sato, et al., J Immunol. 166(6):4185-4194 (2001), Otterbein, et al., Nature medicine, 9:183-190 (2003)). Moreover, low dose CO (reaching <20% COHb saturation) has been demonstrated to be safe and tolerable in Phase 1 and 2 clinical studies and in large numbers of preclinical studies (Rhodes, et al., PLoS One, 7(9): p. e46479 (2012), Purushottam, et al., Sepsis, Acute Respiratory Distress Syndrome, and Acute Lung Injury, D36, A5768-A5768 (2014); Pecorella, et al., Lung Cellular and Molecular Physiology, 309, L857-L871 (2015), Mahan, et al., PloS One 7:e41982 (2012); Ghanizada, et al., Cephalalgia, 38(13): 1940-1949 (2018)). Initial research focused on the delivery of CO gas by inhalation (iCO) (Ryter & Choi, Korean J Intern Med, 28(2): p. 123-40 (2013); Fredenburgh, et al., JCI Insight, 3(23): e124039 (2018); Bathoorn, et al., The European Respiratory Journal, 30:1131-7 (2007); Rosas, et al., Chest, 153(1):94-104 (2018); Misra, et al., Artif Organs, 38:702-7 (2014); Abu Jawdeh, et al., Clin Transplant, 32: e13155 (2018); Misra, et al., Rev Bras Hematol Hemoter., 39(1): 20-27 (2017); Howard, et al., American Society of Hematology, 2014; Misra, et al., EHA Library, 181905; P618 (2017); Dhar, et al., Neurocrit Care, 27:341 (2017); U.S. National Institutes of Health. Dec. 14 2020. ClinicalTrials.gov Retrieved from: clinicaltrials.gov; Wilbur, et al., Agency for Toxic Substances and Disease Registry, 2012, retrieved from ncbi.nlm.nih.gov/books/NBK153693/). However, iCO is difficult to dose owing to the variability of patient ventilation, mask and cannula compliance, and pulmonary absorption associated with the variable capacity of the lung (Gomperts, et al., Am J Hematol, 92(6): p. 569-582 (2017)). Inhaled CO also presents the risk of accidental inhalation exposure of healthcare workers, patients, and families arising from leaks from gas cylinders and other equipment. The use of CO Releasing Molecules (CORM) is also problematic given the well-known toxicity of the carrier molecules, challenging release kinetics, and poor PK/PD (Motterlini & Otterbein, Nat Rev Drug Discov., 9:728-743 (2010), Winburn, et al., Basic & clinical pharmacology & toxicology, 111:31-41 (2012), Natanson, et al., JAMA 299:2304-12 (2008)).

Thus, there is an urgent need for safer and more effective CO therapies to prevent or treat DR in its earlier stages, and to treat DR more effectively in its later stages.

It is an object of the invention to provide improved formulations for use in the prevention and treatment of ocular diseases.

It is also an object of the invention to provide methods for treating an ocular disease in a subject in need thereof.

### SUMMARY OF THE INVENTION

Liquid formulations containing carbon monoxide (CO) dissolved in an amount sufficient for treating an ocular disease, and methods of use thereof for treatment of a subject in need thereof are provided.

In general, the dissolved CO is present, and administered to the subject, in an amount effective to prevent or treat at least one clinical symptom of an ocular disease. Exemplary ocular diseases to be treated or prevented by the present formulations and methods include, but are not limited to, neovascularization of the retina, neovascularization of the choroid, neovascularization of ocular tumors, diabetic retinopathy, retinopathy of prematurity, retinoblastoma, neovascularization of the cornea, sickle cell retinopathy and macular degeneration. In some embodiments, the disease or disorder is characterized by subfoveal choroidal neovascularization.

In some embodiments, the methods include enterally, for example orally, administering a paste, gel, foam, emulsion, Newtonian liquid, or non-Newtonian liquid in which CO is dissolved, e.g., the formulation is made as a paste, gel, foam, emulsion, Newtonian or non-Newtonian liquid in which CO is dissolved. In some embodiments, the CO is dissolved in a carrier containing water and/or oil, such as the drug product HBI-002.

The methods typically include administering a therapeutically effective dose of the liquid CO formulation to the subject. In some forms, the methods include providing a dose sufficient to achieve at least 2%, at least 3%, at least 4%, at least 5%, at least 6%, at least 7%, at least 8%, at least 9%, or at least 10%, up to about 12%, 13%, 14%, or 15% Carboxyhemoglobin (COHb) saturation in the blood.

In some embodiments, the liquid CO formulation contains dissolved CO at a concentration of about 30 milligrams per liter (mg/L) to 4,400 mg/ml or about 400 mg/L. In exemplary embodiments, the methods include enteral administration, for example, orally, rectally, or gastrically. A single dose may be, for example, about 2 mg CO/kg. In some embodiments, the methods include administering a double dose of about 4 mg CO/kg split into two equal doses of about 2 mg CO/kg delivered at about 60 minutes apart. In some embodiments, the methods include administering a single dose of 2 mg/kg, then daily for 7 days with two doses of 2 mg/kg 60 min apart, followed by a single dose per day for up to about 20 days, for a total of about 28 days of drug treatment. In some embodiments, the methods include administering a single dose of 2 mg/kg daily for 6 months. In some embodiments, the methods include orally, rectally, gastrically, or sublingually administering a daily, bi-weekly, or weekly dose of the liquid formulation on an acute, sub-chronic, or chronic basis.

### BRIEF DESCRIPTION OF THE DRAWINGS

The accompanying drawings illustrate several non-limiting embodiments of the disclosed method and compositions and together with the description, serve to explain the principles of the disclosed method and compositions.

FIG. 1 is a bar graph showing the peak blood COHb levels in mice following either a single or double oral dose of HBI-002. Baseline value is pre-treatment; n=3. Single dose was one oral dose of HBI-002 at 2 mg CO/kg; n=3; peak at 10 minutes after dosing. Double dose is a total dose of 4 mg CO/kg split into two equal doses of 2 mg CO/kg each administered 60 min apart; n=2; peak at 5 minutes after the second dose.

FIG. 2 is a bar graph illustrating the effect of HBI-002 treatment on the optokinetic tracking response in a mouse model of retinal ischemia-reperfusion (I/R) injury. Optokinetic tracking response was measured in IR-injured and sham-operated mice treated with vehicle or HBI-002 seven days after ischemic insult using the OptoMotry system. Data are shown as mean±SD where * p<0.05 significance (n=5 per group).

FIG. 3 is a bar graph illustrating the effect of HBI-002 treatment on the histopathology in a mouse model of I/R injury. Shown in FIG. 3 is the morphometric analysis of retinal cryosections to measure total retinal thickness. Data are shown as mean±SD where * p<0.05 significance (n=5). GCL, ganglion cell layer; INL, inner nuclear layer; ONL, outer nuclear layer; RPE, retinal pigment epithelium.

FIG. 4 is a bar graph illustrating the effect of HBI-002 treatment on retinal ischemia-reperfusion (I/R) injury-induced ganglion cell loss in mice. Shown in FIG. 4 is a quantification of RBPMS-positive cells. Data are expressed as cell number/100 μm retinal length and presented as a percent change from sham. Data are shown as mean±SD where * p<0.05 significance (n=5).

FIG. 5 illustrates changes in Heme Oxygenase-1 (HO-1) expression in the retina of vehicle and HBI-002 treated I/R mice. FIG. 5 (top panel) is a bar graph showing quantification of HO-1 expression. The results were normalized to β actin levels and expressed as fold change. Data are shown as mean±SD where * p<0.05 significance (n=5). FIG. 5 (lower panel) are representative immunoblotting images of retinal cryosections from IR-injured and sham-operated eyes of vehicle or HBI-002 treated mice 7 days after ischemic insult to measure HO-1 protein levels.

FIGS. 6A-6C are bar graphs showing the body weight (FIG. 6A), blood glucose (FIG. 6B), and visual acuity (FIG. 6C) in 20 weeks control, diabetic mice, and HBI-002-treated (2 mg/kg or 10 ml/kg 5×/week) mice.

FIGS. 7A-7H are bar graphs of the results from OCT analysis performed in anesthetized mice, and the thickness of the total retina and various retinal layers calculated. Shown are the total thickness (FIG. 7A), retinal nerve fiber layer (RNFL, FIG. 7B), inner plexiform layer (IPL, FIG. 7C), inner nuclear layer (INL, FIG. 7D), outer plexiform layer (OPL, FIG. 7E), outer nuclear layer+photoreceptor inner segment (ONL+IS, FIG. 7F), photoreceptor outer segment (OS, FIG. 7G), and retinal pigment epithelium (RPE, FIG. 7H).

FIGS. 8A-8D are line graphs of the average scotopic a-wave and b-wave amplitudes for non-diabetic and diabetic (DB) mice treated with vehicle or HBI-002 20 weeks post-diabetes induction.

### DETAILED DESCRIPTION OF THE INVENTION

The disclosed method and formulations may be understood more readily by reference to the following detailed description of particular embodiments and the Examples included therein and to the Figures and their previous and following description.

### I. Definitions

As used herein, “liquid” is given its broadest reasonable meaning, and thus includes both Newtonian liquids and non-Newtonian liquids. “Liquid” thus includes formulations in which the main component, by weight, is a liquid. The term “liquid” thus includes pastes, gels, and emulsions. It likewise includes foams in which CO bubbles are entrapped. For ease of reference, within the present disclosure, reference is made to “liquids” in which CO is “dissolved”. However, it is to be understood that such references are not limited to Newtonian liquids in which CO is in solution, but rather to all liquids, pastes, gels, emulsions, foams, etc. in which gaseous CO is dissolved, entrapped, and so on.

The term, “in vitro” refers to an artificial environment and to processes or reactions that occur within an artificial environment. In vitro environments include, but are not limited to, in solution or suspension, and cell cultures.

The term “in vivo” refers to in or associated with an organism, such as an animal.

As used herein, the terms “subject,” “individual,” and “patient” refer to any individual who is the target of treatment using the disclosed formulations. The subject can be a vertebrate, for example, a mammal. Thus, the subject can be a human. The subjects can be symptomatic or asymptomatic. The term does not denote a particular age or sex. Thus, adult, aged adult, and newborn subjects, whether male or female, are intended to be covered. A subject can include a control subject or a test subject.

As used herein, “treat” means to prevent, reduce, decrease, or ameliorate one or more symptoms, characteristics or comorbidities of an ocular disease, disorder or condition; to reverse the progression of one or more symptoms, characteristics or comorbidities of an ocular disorder; to halt the progression of one or more symptoms, characteristics or comorbidities of an ocular disorder; to prevent the occurrence of one or more symptoms, characteristics or comorbidities of an ocular disorder; to inhibit the rate of development of one or more symptoms, characteristics or comorbidities or combinations thereof.

As used herein, the term “effective amount” or “therapeutically effective amount” means a dosage sufficient to treat, inhibit, or alleviate one or more symptoms of a disease state being treated or to otherwise provide a desired pharmacologic and/or physiologic effect. The effective amount required will vary from subject to subject, depending on subject-dependent variables (e.g., physical conditions, age, sex, immune system health, species, and weight of the subject), the disease state, and the treatment being effected.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients. By “pharmaceutically acceptable” is meant a material that is not biologically or otherwise undesirable, i.e., the material can be administered to a subject along with the selected agent without causing any undesirable biological effects or interacting in a deleterious manner with any of the other components of the pharmaceutical composition in which it is contained.

As used herein, the term “pharmaceutically-acceptable carrier” means one or more compatible solid or liquid fillers, diluents or encapsulating substances which are suitable for administration to a human or other vertebrate animal.

The use of the terms “a,” “an,” “the,” and similar referents in the context of the disclosure including the claims are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### II. Formulations

Provided are formulations containing carbon monoxide (CO) dissolved in an amount sufficient for treating an ocular disease in a subject in need thereof. The formulations contain a tunable amount of CO that is not bound to a carrier molecule (i.e. not a CORM). Typically, the enterally, for example orally, administered formulation can be efficiently absorbed from the gastrointestinal tract. A preferred example, referred to herein as HBI-002, is an aqueous drug product containing CO (Belcher, et al., PLoS One, 13(10): p. e0205194 (2018)), and is suitable for enteral, including oral, administration. See, also e.g., U.S. Pat. Nos. 9,980,981 and 10,716,806 which are specifically incorporated by reference herein in their entireties.

In general, the dissolved CO is present in an amount sufficient to prevent or treat at least one clinical symptom of an ocular disease when administered to a subject in need thereof. Various ocular diseases and disorders treatable by the disclosed CO formulation are discussed below.

In some embodiments relating to aqueous compositions, the concentration of dissolved CO will generally not exceed 800 mg/l. For example, an effective concentration for convenient dosing of CO can be between about 100 mg/l and 800 mg/l, although concentrations between about 50 mg/l and about 100 mg/l are also suitable for patients amenable to intake of relatively large volumes of liquid during treatment periods. Although the skilled artisan will immediately understand that all particular values within the range of about 30 mg/l and about 800 mg/l are specifically contemplated by this application, the following concentration values, and the various ranges defined by the collection of specific values, provide convenient reference points for the practitioner to develop compositions: 30 mg/l, 35 mg/l, 40 mg/l, 45 mg/l, 50 mg/l, 55 mg/l, 57 mg/l, 60 mg/l 65 mg/l, 70 mg/l, 75 mg/l, 78 mg/l, 80 mg/l, 85 mg/l, 90 mg/l, 95 mg/l, 100 mg/l, 105 mg/l, 110 mg/l, 115 mg/l, 120 mg/l, 125 mg/l, 130 mg/l, 135 mg/l, 140 mg/l, 145 mg/l, 150 mg/l, 155 mg/l, 160 mg/l, 165 mg/l, 170 mg/l, 175 mg/l, 180 mg/l, 190 mg/l, 200 mg/l, 210 mg/l, 225 mg/l, 250 mg/l, 275 mg/l, 300 mg/l, 325 mg/l, 350 mg/l, 375 mg/l, 400 mg/l, 425 mg/l, 450 mg/l, 475 mg/l, 500 mg/l, 525 mg/l, 550 mg/l, 575 mg/l, 600 mg/l, 625 mg/l, 650 mg/l, 675 mg/l, 700 mg/l, 725 mg/l, 750 mg/l, 750 mg/l. Aqueous compositions can include any specific concentration value between 30 mg/l and 800 mg/l, can include any specific concentration range between 30 mg/l and 800 mg/l, or can contain a concentration of dissolved CO of at least any of the concentration values between 30 mg/l and 800 mg/l.

In some embodiments relating to lipid/oil/fat-based compositions (discussed in more detail below), the concentration of dissolved CO will generally not exceed 4,400 mg/l. For example, an effective concentration for convenient dosing of CO can be between about 500 mg/l and 4,400 mg/l, such as between about 500 mg/l and 4,000 mg/l, although concentrations between about 75 mg/l and about 500 mg/l are also suitable for patients amenable to intake of relatively large volumes of such liquids during treatment periods. Likewise, concentrations on the lower end of these ranges, as well as higher and lower than these ranges, are also contemplated. Although the skilled artisan will immediately understand that all particular values within the range of about 30 mg/l and about 4,400 mg/l are specifically contemplated by this application, the following concentration values, and the various ranges defined by the collection of specific values, provide convenient reference points for the practitioner to develop compositions: 75 mg/l, 100 mg/l, 125 mg/l, 150 mg/l, 175 mg/l, 200 mg/l, 225 mg/l, 250 mg/l, 275 mg/l, 300 mg/l, 325 mg/l, 350 mg/l, 375 mg/l, 400 mg/l, 425 mg/l, 450 mg/l, 475 mg/l, 500 mg/l, 525 mg/l, 550 mg/l, 575 mg/l, 600 mg/l, 625 mg/l, 650 mg/l, 675 mg/l, 700 mg/l, 725 mg/l, 750 mg/l, 775 mg/l, 800 mg/l, 825 mg/l, 850 mg/l, 875 mg/l, 900 mg/l, 925 mg/l, 950 mg/l, 975 mg/l, 1000 mg/l, 1025 mg/l, 1050 mg/l, 1075 mg/l, 1100 mg/l, 1125 mg/l, 1150 mg/l, 1175 mg/l, 1200 mg/l, 1225 mg/l, 1250 mg/l, 1275 mg/l, 1300 mg/l, 1325 mg/l, 1350 mg/l, 1375 mg/l, 1400 mg/l, 1425 mg/l, 1450 mg/l, 1475 mg/l, 1500 mg/l, 1525 mg/l, 1550 mg/l, 1575 mg/l, 1600 mg/l, 1625 mg/l, 1650 mg/l, 1675 mg/l, 1700 mg/l, 1725 mg/l, 1750 mg/l, 1775 mg/l, 1800 mg/l, 1825 mg/, 1850 mg/l, 1875 mg/l, 1900 mg/l, 1925 mg/l, 1950 mg/l, 1975 mg/l, 2000 mg/l, 2050 mg/l, 2100 mg/l, 2150 mg/l, 2200 mg/l, 2250 mg/l, 2300 mg/l, 2350 mg/l, 2400 mg/l, 2450 mg/l, 2500 mg/l, 2550 mg/l, 2600 mg/l, 2650 mg/l, 2700 mg/l, 2750 mg/l, 2800 mg/l, 2850 mg/l, 2900 mg/l, 2950 mg/l, 3000 mg/l, 3100 mg/l, 3200 mg/l, 3300 mg/l, 3400 mg/l, 3500 mg/l, 3600 mg/l, 3700 mg/l, 3800 mg/l, 3900 mg/l, 4000 mg/l, 4100 mg/l, 4200 mg/l, 4300 mg/l, and 4400 mg/l. Lipid/oil/fat compositions can include any specific concentration value between 30 mg/l and 4400 mg/l, can include any specific concentration range between 30 mg/l and 4400 mg/l, or can contain a concentration of dissolved CO of at least any of the concentration values between 30 mg/l and 4400 mg/l.

The liquid composition is not particularly limited in its components, although exemplary embodiments disclosed herein have shown to be superior in the amount of CO that can be dissolved. While not so limited in all embodiments, in exemplary embodiments, the liquid composition is a water-based composition. It is to be understood that the term “water-based composition” includes all compositions including water as a solvent, including, but not limited to: compositions in which water is the sole solvent; water-oil mixtures (e.g., water-in-oil and oil-in-water emulsions); aqueous solutions, suspensions, colloids, and dispersions; water-alcohol mixtures; and combinations of these.

In some embodiments, the CO composition includes one or more FDA-defined Generally Recognized as Safe (GRAS) components. In some embodiments, the composition is manufactured using a controlled, reproducible process to achieve the targeted CO concentration.

It has been found that aqueous compositions including one or more “complex” components provides superior CO-dissolving capacity. “Complex” components, as used herein, are substances that are polymeric in nature, biologic in nature, such as those derived from fatty acids, or otherwise include at least one bonding interaction site for CO. Interactions can be physical (e.g., hydrophobic, Van der Waals), or chemical (e.g., ionic or covalent). Examples of complex components include, but are not limited to: proteins, polypeptides, and peptides; polysaccharides; lipids, fats, and oils; and alcohols having two or more carbon atoms. In some embodiments, lipid, protein, or both are present in the compositions. In these embodiments, it is preferable that the combined amount of protein and lipid be greater than 5% (w/v), and even as high as 40% lipid and protein, or higher. In some formulations, the composition includes greater than 5% lipid and greater than 5% protein.

The precise chemical structures of the complex components are not particularly limited. Rather, it is sufficient that the complex components function to assist in increasing the solubility of CO in the composition. However, to aid the practitioner in selecting appropriate complex components, the following is a non-limiting listing of types of complex components: proteins and fats/oils/lipids/triglycerides of animal derivation, such as those in milk; proteins and fats/oils/lipids/triglycerides of plant derivation; mono-, di-, and poly-saccharides; vitamins; natural and artificial sweeteners; and natural and artificial flavorings. Any and all of the various molecules that are encompassed within these groups can be included as part of the present formulations.

In embodiments, the liquid composition takes the form of a beverage for oral consumption. Non-limiting examples of beverages are: bottled water, such as fruit- or berry-flavored waters; dietary/nutritional supplements, such as those formulated for infants and young children (e.g., baby formula, such as Similac® (Abbott, Abbott Park, Ill.) and Enfamil® (Mead Johnson & Company, Glenview, Ill.)) or adults (e.g., Ensure® (Abbott, Abbott Park, Ill.), and Peptamen® and Nutren® (Nestle, Vevey, Switzerland)); liquid dairy or dairy-based products, such as milk, cream, yoghurt, or a milkshake; liquid soy or soy-based products, such as soy milk or soy yoghurt; liquid rice or rice-based products, such as rice drinks; sports drinks or dietary supplements, such as whey protein based drinks and Gatorade® (Pepsico, Purchase, N.Y.); coffee-based drinks, such as those supplemented with dairy products; and sugar-containing or sugar-free sodas. As discussed in more detail below, certain liquid compositions are supersaturated with CO at room temperature and atmospheric pressure. As such, certain beverages can be effervescent as a result of release of a portion of the supersaturated CO. This effervescent property can enhance the patient's experience when ingesting the beverage, and can improve compliance with a dosing regimen.

In some embodiments, the liquid composition takes the form of a foam- or gel-based food product. For example, in some embodiments, the liquid composition is provided in the form of a gel, such as a gelatin or pudding, such as those commercially available under the Jell-O® brand (KraftFoods, Inc., Glenview, Ill.). Yet again, in some embodiments, the liquid composition is provided in the form of a foam, such as those commercially available under the Coolwhip® brand (KraftFoods, Inc., Glenview, Ill.) and ReddiWip® brand (ConAgra Foods, Inc., Omaha, Nebr.).

A beverage or food product can be provided in a container. In preferred embodiments, the container is a sealable container of the type widely used for providing commercial beverages and food products to the public. Non-limiting examples of sealable containers for holding the beverage and/or food product are: plastic bottles with twist on/off tops; aluminum cans with pop tops; glass bottles with twist on/off tops; and glass bottles with crimp-sealed aluminum tops or tops made of other pliable metals. For convenience in delivering an effective amount of CO to a subject in need, in preferred embodiments, the amount of beverage or food product in a single container is an adequate volume of beverage or food product to supply a single dose of CO (e.g., 5 ml, 10 ml, 30 ml, 50 ml, 75 ml, 100 ml, 150 ml, 177 ml, 180 ml, 237 ml, 300 ml, 355 ml, 500 ml, one liter). As such, it is recognized that the liquid compositions can be provided such that a daily dosage for treatment of a disease or disorder (e.g., a symptom thereof) is conveniently provided in volumes of about 3 liters or less, such as 2.5 liters, 2 liters, 1.8 liters, 1.5 liters, 1 liter, 330 ml, 300 ml, 180 ml, 30 ml, or less. However, it is to be understood that, in situations where the beverage or food product is supplied in a re-sealable container (e.g., a bottle with a twist on/off cap), the amount of beverage or food product in the container can represent more than one dose of CO.

The products can be products typically associated with medical procedures, such as solutions and containers containing solutions (e.g., IV bags). Alternatively, the products can be in the form of more easily administered products, such as canned or bottled solutions or foods. The technology is simple to apply, unlikely to be associated with significant side effects, and acceptable to the affected population, thus resulting in reliable utilization of the treatment method.

In summary, in various exemplary embodiments, provided is a liquid composition including dissolved gaseous carbon monoxide (CO) in an amount of from 30 mg/l to 4400 mg/l in the liquid composition. In embodiments, the composition includes dissolved gaseous CO in an amount of from 50 mg/l to 400 mg/l, from 75 mg/l to 750 mg/l, and from 550 mg/l to 4400 mg/l. In general, the dissolved CO is present in an amount sufficient to prevent or treat at least one clinical symptom of an ocular disease or disorder affected by CO. Various diseases and disorders treatable with the composition are discussed below. In embodiments, in addition to dissolved gaseous CO, the composition further includes at least one of: protein, lipid, fat, triglyceride, complex carbohydrate, sugar, sugar substitute, fruit juice, carbohydrate, cellulose, fiber, citric acid, artificial flavoring, natural flavoring, gum, pectin, ascorbic acid, preservative, saponin, oil, oil emulsion, pH buffer, and a salt. For the liquid portion of the composition, in exemplary embodiments, water, ethanol, or both are used. In embodiments, the liquid composition is one in which the amount of dissolved CO is greater than occurs under ambient temperature and pressure and at a pH of 7.0 and/or at pH of 7.0, atmospheric pressure, and 21° C. For example, the dissolved CO can be two or more times the amount dissolved under ambient temperature and pressure and at a pH of 7.0 and/or at pH of 7.0, atmospheric pressure, and 21° C.

As mentioned above, the liquid composition can include dissolved gaseous CO, or gaseous CO entrapped in bubbles, in an amount of at least 0.03 grams of gas per kilogram of water or other liquid (i.e., at least 30 mg/l). In embodiments, the amount of dissolved or entrapped CO is greater than 0.04 grams of gas per kilogram of water or other liquid. In embodiments, the amount of dissolved CO is equal to or greater than the amount that occurs under two atmospheres of pressure at 10° C. and at a pH of 7.0. In certain embodiments, the liquid composition is one in which dissolved or entrapped CO can be administered enterally, for example orally, or otherwise through the gastrointestinal tract, or intravenously. In some embodiments, the composition does not contain constituents other than potentially CO, that are toxic at the dosage administered.

The disclosed methods typically include administering a CO-containing liquid composition to a patient in need thereof. Although the method may include performing the administering step a single time, the step of administering can also be repeated any number of times. Indeed, in preferred embodiments, the step of administering is repeated a sufficient number of times to achieve a carbonmonoxy-hemoglobin (herein referred to as “CO-Hb”) concentration suitable for the ocular disease or disorder being treated. For example, in some embodiments, an average CO-Hb concentration of between 3% and 15%, more preferably between 3% and 12%, most preferably between 4% and 11%, such as about 5% to 10%, is desirable. It is known that the half-life of the alpha-phase of CO in the human bloodstream is about 4-6 hours. Therefore, an average CO-Hb concentration in the bloodstream can be achieved, for example, through a dosing regimen of four or fewer doses per day, preferably equally spaced, such as four doses per day (i.e., every six hours), three doses per day (i.e., one dose every eight hours), two doses per day (i.e., every twelve hours), or one dose per day. Due to the relatively high CO concentration achievable in the disclosed liquid compositions, relatively small volumes of liquid composition can be administered per dose or per day, such as: 2 liters per day, 1.5 liter per day, 1 liter per day, 0.7 liter per day, 0.5 liter per day, 0.25 liter per day, 0.1 liter per day, 0.05 liter per day, and 0.01 liter per day, and other amounts disclosed herein. Dosing of small volumes improves patient compliance and provides an overall superior outcome for the patient. As there is no known detrimental effect to long-term exposure to CO at these levels, the daily administration can be performed indefinitely.

Delivery of CO to a patient via administration of liquid is most conveniently achieved using generally available containers, such as a metal can or glass or plastic bottle. The liquid and type of container is not particularly limited, with the exception that the liquid must allow for adequate levels of CO to dissolve, while the container must be fabricated of material that is sufficiently impermeable to CO and sufficiently pressure resistant. Of course, the practitioner will need to adjust the composition of the liquid in some situations to optimize CO dissolving into the liquid and thus absorption into the body of the patient. For example, the salinity, pH, sugar content, amount of organic compounds (e.g., alcohol), protein content, lipid content, etc. can be varied to optimize the taste, consistency, etc. of the composition, and the amount of CO that dissolves into the liquid and also to optimize CO absorption once delivered to the patient. Also, the pressure, temperature, and components in the composition during CO dissolution can be varied to optimize the amount of CO that dissolves into the liquid. Likewise, non-aqueous solutions of various constituents can be used, as well as aqueous or non-aqueous compositions that include undissolved constituents.

Among the many containers that can be used, mention may be made of: glass bottles, plastic bottles; and aluminum cans, and containers fabricated from combinations of glass, plastic, and aluminum, steel, and other metals.

CO has a number of medical properties and has shown promise in treating a variety of diseases. The primary challenge in using CO as a therapeutic given its potential toxicity is to deliver a small but sufficient amount of CO to treat disease without causing harm. Until now, it has not proven possible to do this.

There are a number of significant limitations to using the delivery of CO dissolved in liquid and used through the GI tract or IV for therapeutic or prophylactic purposes. These limitations have been overcome with the disclosed formulations. The primary barrier to date has been delivering sufficient quantities of CO. The solubility of CO in aqueous solutions is low at room temperature and normal atmospheric pressure (ATM). Thus, using approaches previously proposed in the art, which rely on dissolving CO into an aqueous composition at room temperature and at atmospheric pressure, the volume of liquid necessary to deliver a therapeutic dose of CO is logistically prohibitive. However, using disclosed materials and methods, therapeutically useful levels of CO can be achieved.

For example, CO can be dissolved in an aqueous liquid under higher than ambient pressure. The use of pressure to dissolve the CO increases the achievable concentration of CO in solution. Tables 1 and 2 demonstrate the significantly increased CO concentrations achievable under pressure and the corresponding substantial decrease in the necessary amount of aqueous solution needed to provide an effective dose of CO. For example, lowering of the temperature below 2° C. and increasing the pressure above 10 ATM can achieve an even greater concentration of CO dissolved in the aqueous solution. The values presented in Table 1 are for demonstration purposes only. Although raising the pressure above 10 ATM might not be commercially practicable, it is technologically feasible at this time.

TABLE 1

| | CO Concentration Achievable in |
|---|---|
| Temperature, Pressure | Aqueous Composition (mg/L water) |
| 21° C.; 1 ATM | 25 |
| 2° C.; 1 ATM | 40 |
| 2° C.; 2 ATM | 80 |
| 2° C.; 3 ATM | 120 |
| 2° C.; 5 ATM | 200 |
| 2° C.; 10 ATM | 400 |

Table 1 demonstrates that under greater than ambient pressure and low temperature, it is possible to achieve a CO concentration in solution that enables a therapeutic dose of CO in a volume of aqueous solution that can reasonably be taken orally by a patient in a 24-hour period. The volumes needed to be administered per day are well within amounts taken daily by many people. Thus, compliance by patients should be vastly improved as compared to any attempts at treatment with CO-containing liquids known in the art.

The use of a GI formulation (e.g., a formulation for enteral administration) overcomes a challenge of using a CO-infused aqueous composition as a therapeutic. An aqueous oral or GI formulation allows the use of a solution with CO dissolved under higher than ambient pressure and low temperature, providing a therapeutic dose of CO. When CO is dissolved into solution under greater than ambient pressure, and the solution is then moved into an ambient pressure environment upon delivery of the solution, the CO gas will bubble out of solution (similar to liquid solutions containing dissolved CO₂). Bubbles of gas pose a substantial danger to a patient in many delivery mechanisms, such as IP and intravenous delivery, amongst others. However, the bubbling of CO gas out of solution in the stomach or intestine does not pose a substantial safety risk. Thus, the use of a delivery route that allows the bubbling of CO out of solution allows for the treatment of therapeutic indications that require a high CO concentration. Also, dissolving CO into solution under temperatures close to or below 0° C. increases the CO saturation of the solution, as compared to dissolving CO at room temperature. The administration of a very cold solution poses discomfort and danger to a patient in many delivery mechanisms, such as IP and intravenous delivery, amongst others. However, the use of a cold liquid in the stomach or intestine does not pose a substantial safety risk or substantial discomfort. Thus, the use of a delivery route that allows the administration of a cold solution allows for the use of these composition in therapeutic indications that require a high CO concentration. Furthermore, dissolving CO into a liquid at relatively low temperatures is contemplated, the CO need not be delivered to a patient by way of a cold liquid. For example, a CO-containing liquid composition can be prepared in a sealable container. Once sealed, the container need not be maintained at a cold temperature for the CO to stay in solution. Although a greater amount of CO will escape from solution if the container is opened at higher temperatures than at lower temperatures, ingestion (or other delivery) of the liquid to the patient in a relatively short time period after opening of the container will minimize loss of CO from the liquid.

Additionally, complex liquid compositions are superior to water or relatively simple aqueous compositions for therapeutic delivery of CO. Conventional attempts at delivering CO to cells used CO dissolved in water or in aqueous solutions of water and salts. In preferred embodiments, the liquid composition for delivery of CO is an aqueous composition that contains one or more relatively complex molecules, such as proteins, lipids, oils, alcohols, and/or carbohydrates. It has been found that the presence of these complex molecules allows greater CO solubility as compared to compositions lacking them. As such, inclusion of these complex molecules overcomes a challenge of using CO as a therapeutic. Preferred embodiments thus include administration of complex aqueous compositions including therapeutic concentrations of CO mixed with one or more lipids, proteins, or other substances that aid in increasing the concentration of CO in the aqueous composition.

Methods of making a liquid composition containing CO dissolved in a treatment-effective amount are discussed in U.S. Pat. Nos. 9,980,981 and 10,716,806 and provided herein. Previous attempts in the art to create a liquid composition containing dissolved gaseous CO involved dissolving CO in an aqueous liquid at ambient temperature and atmospheric pressure, resulting in a composition that is unsuitable for use as an in vivo therapeutic or prophylactic agent due to the low CO content in the liquid. In contrast, this disclosed preparation methods can include the use of high pressure, cold temperature, or a combination of the two. Preferably, the method also includes the use of a liquid composition that includes one or more complex components, which aid in increasing the concentration of CO dissolved in the composition.

The method of making a CO-containing liquid composition typically includes subjecting a liquid composition to a high pressure while exposing the composition to gaseous CO for a sufficient amount of time to achieve an adequately high concentration of CO in the liquid composition to provide a therapeutically effective and/or prophylactically-effective composition. However, it is to be noted that, in certain embodiments in which the compositions include lipids, fats, or oils, introduction of therapeutic levels of CO might not require the use of higher than atmospheric pressure. In preferred embodiments, the step of exposing includes infusing CO into the liquid composition by bubbling through a cannula, aerator, or other equivalent device or method. In some embodiments, the liquid composition is subjected to mixing or stirring during the process of exposing to CO in order to facilitate dissolving of the CO into the liquid composition.

While not required, in embodiments relating to commercial production of the liquid composition, it is preferred that the CO-containing liquid composition be sealed in a CO-impermeable container to preclude loss of dissolved CO over time. Accordingly, in embodiments, the method of making a CO-containing liquid composition includes dispensing the liquid composition into a sealable CO-impermeable container and sealing the container after an appropriate amount of CO has been dissolved in the liquid composition. Dispensing of the liquid can be performed before, during, or after dissolving CO into the liquid composition. Typically, sealing will be performed under CO gas and under greater than atmospheric conditions to minimize loss of dissolved CO during the process of sealing. As such, it is preferable that the container and sealing mechanism (e.g., cap, top) are resistant to the pressures used during dissolving of CO into the composition (e.g., from about 1.1 ATM to about 8 ATM or higher). As discussed above, any number of sealable containers and caps are known in the art of bottling and canning of liquids, and any of these can be used within the context of the present formulations.

The method of making a CO-containing liquid composition relies on the use of greater than atmospheric pressure (i.e., “high” pressure) to increase the amount of CO dissolved in the liquid composition. In some embodiments, at least 1.1 atmospheres (ATM) of pressure is used in the process of introducing CO into the liquid composition. Preferably, where high pressure is used, at least 1.2 ATM is used. In certain exemplary embodiments, 2 ATM, 3 ATM, or 5 ATM is used during introduction of CO into the liquid composition. It is contemplated that pressures above 5 ATM are also suitable, such as 6 ATM, 7 ATM, 8 ATM, and 10 ATM or higher. In accordance with the discussion concerning ranges set forth above, all specific values, and all possible ranges, falling within the atmospheric conditions discussed herein are contemplated. Furthermore, variations in the source of CO, the equipment used to introduce the CO into the liquid composition, the pressure of the CO being used (i.e., volume supplied per unit time), and the atmospheric pressures used will affect the amount of time required to achieve a suitable concentration of CO in the composition.

The method of making a CO-containing liquid composition preferably includes subjecting the composition to a low temperature during the step of exposing the liquid to CO. It has been found that lowering the temperature from ambient room temperature (about 21° C.) to about 2° C. increases the amount of CO that is dissolved in the liquid composition. A low temperature is typically a temperature at or below 4° C., preferably at or below 2° C., such as 1° C., 0° C., −1° C., −2° C., −3° C., −4° C., −5° C., −6° C., −10° C., −12° C., −14° C., −16° C., −18° C., −20° C., or below. In embodiments where the composition is exposed to a low temperature, it is preferred that the CO-containing liquid composition be sealed to preclude loss of dissolved CO over time. Accordingly, in embodiments, the method of making a CO-containing liquid composition includes dispensing the liquid composition into a sealable container and sealing the container after an appropriate amount of CO has been dissolved in the liquid composition. Dispensing of the liquid can be performed before, during, or after dissolving CO into the liquid composition. Typically, sealing will be performed under low temperature to minimize loss of dissolved CO during the process of sealing. As discussed above, any number of sealable containers and caps are known in the art of bottling and canning of liquids, and any of these can be used within the context of the present disclosure. For the sake of clarity, in embodiments where both high pressure and low temperature are used, it is preferred that sealing be performed under both high pressure and low temperature, although the same pressure and temperature used for dissolving CO into the liquid need not be used. Further, it is to be understood that, once the CO-containing liquid is sealed in a container under CO gas, it is not necessary to maintain the sealed container at high pressure and low temperature, as the sealed container will not allow dissolved CO to escape from solution.

Thus, three important parameters that can be modulated to achieve a liquid composition having a therapeutically- and/or prophylactically effective amount of dissolved gaseous CO include: introducing gaseous CO under high pressure; introducing gaseous CO under low temperature; and the presence of complex substances in the liquid composition. However, other parameters can be adjusted to improve or otherwise alter the concentration of CO in solution, or simply to alter the overall taste and consistency. These parameters can be adjusted for any number of reasons, including, but not limited to altering the taste of the liquid; altering the sweetness, tartness, or tang of the liquid; altering the pH of the liquid; altering the salinity of the liquid; and altering the consistency of the liquid. It has been found that alterations in many parameters do not significantly affect the overall CO carrying capacity of a liquid composition, with the main exceptions of alteration of pressure, temperature, and presence of complex components, as described above. For example, tests show that, for a given pressure, temperature, and complex component combination, variations in pH, simple sugar concentrations, and salt concentrations have little effect on dissolved CO levels. Accordingly, the practitioner may adjust various parameters to suit a particular need without departing from the concept of the compositions.

Tables 2-4 show data indicating the role of proteins, fats, and other complex components in raising the dissolved CO concentration in liquid compositions.

Table 2 shows that CO can be dissolved in liquid compositions including fats and proteins (Ensure® (Abbott, Abbott Park, Ill.), 10%-50% in water) and standard cream from cow milk (50% cream in water, v/v) at concentrations higher than achievable in water alone, on the order of at least 28 mg/l. Although improved dissolved CO concentrations can be achieved at 1 ATM and 21° C. using low levels of proteins and fats (e.g., 10% Ensure®), the amount of dissolved CO can be increased by increasing the amounts of proteins and fats, and by increasing the gas-phase pressure applied during the dissolving process.

Table 3 shows that the amount of pressure provided during the process of dissolving CO into a water-containing liquid composition is an important factor in achieving a composition with therapeutic levels of CO. More specifically, Table 4 shows that, for a given percent of Ensure® or standard cream, increasing the pressure during dissolving of CO results in a significant increase in the amount of CO infused into the composition.

Table 4 shows a comparison of the amounts of liquid composition needed to provide a therapeutic dose of CO to non-SCD subjects (referred to as “normal” patients) using aqueous compositions and compositions including proteins and fats (i.e., a 50% cream composition). As can be seen from the Table, the volume of composition needed to be administered is one-half or less for liquid compositions including proteins and fats as compared to simple aqueous compositions.

TABLE 2
CO Concentrations with Protein and Fat Containing
Solutions at Various Pressures (Solutions in Water)

| Solution | Temp. | | Pressure | CO Concentration (mg/L) |
|---|---|---|---|---|
| 50% Ensure ® | ~21° | C. | 1 ATM | 60 |
| 50% Ensure ® | ~21° | C. | 2 ATM | 120 |
| 50% Ensure ® | ~21° | C. | 3 ATM | 180 |
| 50% Ensure ® | ~21° | C. | 5 ATM | 300 |
| 50% Ensure ® | ~21° | C. | 10 ATM | 600 |
| 50% Cream | ~2° | C. | 1 ATM | 75 |
| 50% Cream | ~2° | C. | 2 ATM | 150 |
| 50% Cream | ~2° | C. | 3 ATM | 225 |
| 50% Cream | ~2° | C. | 5 ATM | 375 |
| 50% Cream | ~2° | C. | 10 ATM | 750 |

TABLE 3

| | | | | Necessary Daily |
|---|---|---|---|---|
| | | Necessary Daily | | Volume of Non- |
| | | Volume of | CO | Aqueous Solution |
| | CO Concentration | Aqueous Solution | Concentration in | (50% Cream) to |
| | in Aqueous | to Achieve | Non-aqueous | Achieve Average |
| Temperature, | Solution | Average Increase | Solution | Increase of 5% |
| Pressure | (mg/L water) | of 5% CO-Hb | (mg/L water) | CO-Hb |
| 2° C.; 1 ATM | 40 | 8.4 L | 75 | 3.6 L |
| 2° C.; 2 ATM | 80 | 4.2 L | 150 | 1.8 L |
| 2° C.; 3 ATM | 120 | 2.8 L | 225 | 1.2 L |
| 2° C.; 5 ATM | 200 | 1.7 L | 375 | 0.7 L |
| 2° C.; 10 ATM | 400 | 0.8 L | 750 | 0.4 L |

TABLE 4
CO Concentrations with Various Protein and Fat Containing
Solutions (~1 atmosphere pressure; Solutions in Water)

| Solution | Temperature | CO Concentration (mg/L) | N |
|---|---|---|---|
| 10% Ensure ® | ~21° C. | 28.1 | 3 |
| 20% Ensure ® | ~21° C. | 34.2 | 5 |
| 30% Ensure ® | ~21° C. | 43.2 | 4 |
| 40% Ensure ® | ~21° C. | 46.3 | 2 |
| 50% Ensure ® | ~21° C. | 59.8 | 2 |
| 50% Cream | ~21° C. | 63.9 | 10 |
| 50% Cream | ~2° C. | 74.9 | 10 |

### III. Methods of Use

Methods for treating ocular diseases, ocular disorders, and ocular conditions in a subject are provided. The disclosed methods typically include enterally, for example orally, administering a therapeutically effective amount of a disclosed carbon monoxide formulation to a subject in need thereof. As used herein, the term “ocular disease” or “ocular disorder” refers to an abnormal or aberrant medical condition in association with the eyes. Examples include, but are not limited to, glaucoma, age-related macular degeneration (AMD), ischemic retinopathy, optic neuropathy, diabetic retinopathy (DR), diabetic macular edema (DME), uveitis, and senile cataract. The ocular disease may be a disease or disorder associated with oxidative stress and/or hypoxia-induced damages to the eyes, or more particularly to the retinal pigment epithelium (RPE). Examples include, but not limited to, glaucoma, AMD, ischemic retinopathy, optic neuropathy, DR and DME. The ocular disease may be a disease or disorder associated with reduced ocular blood flow. Examples include, but not limited, to glaucoma, ischemic retinopathy, sickle cell retinopathy, DR, retinopathy of prematurity traumatic optic neuropathy, retinitis pigmentosa and AMD.

Additionally or alternatively, in some embodiments, the compositions are administered and/or the methods are used effective to reduce and/or protect a subject from an increase in retinal oxidative stress; improve or reduce body weight and/or prevent weight gain; improve, reduce, or prevent blood glucose (e.g., hyperglycemia); improve, reduce, or protect from an increase in vascular leakage in the retina; improve, reduce, or protect from an increase in retinal thinning; improve, reduce, or protect against reactive gliosis, improve or protect from loss of electroretinogram response; provide neuroprotection; or a combination thereof.

In some embodiments, the subject had diabetes.

A. Exemplary Ocular Diseases to be Treated

Ocular diseases that may be treated or prevented by the present formulations and methods include neovascularization of the retina, neovascularization of the choroid, neovascularization of ocular tumors, diabetic retinopathy, retinopathy of prematurity, retinoblastoma, neovascularization of the cornea, and macular degeneration. More specifically, suitable diseases include those that exhibit subfoveal choroidal neovascularization, including pathological myopia and exudative age-related macular degeneration. Pathological myopia can be referred to alternately as proliferative myopathy or myopic macular degeneration. As used herein, the terms pathological myopia, proliferative myopathy and myopic macular degeneration all refer to the same disease state. Ocular tumors may include retinoblastoma, primary ocular lymphoma, choroidal melanoma, and intraocular melanoma.

1. Diabetic Ocular Disease

In some forms, the subject to be treated is suffering from a diabetic ocular disease. Diabetic ocular disease typically occurs in individuals suffering from diabetes mellitus. Many diabetics notice blurred vision when their blood sugar is particularly high or low. This blurred vision results from changes in the shape of the lens of the eyes, and usually reverse when their blood sugar returns to normal. Diabetes is a disease that affects not only the patient's blood sugar levels, but also the blood vessels. Symptoms associated with diabetes (including elevated blood pressure) cause damage to the microcirculatory system including the capillaries associated with the retina. Capillary damage results in a decreased flow of blood to isolated regions of the retina. In addition, the damaged blood vessels tend to leak, which produces swelling within the retina.

There are two main categories of diabetic eye disease. The first category is termed background diabetic retinopathy or non-proliferative retinopathy. This is the earliest stage of diabetic retinopathy (DR). DR is characterized by damage to small retinal blood vessels which results in the effusion of fluid (blood) into the retina. Most visual loss during this stage is due to the fluid accumulating in the macula. This accumulation of fluid is called macular edema and can cause temporary or permanent decreased vision. The second category of diabetic retinopathy is termed proliferative diabetic retinopathy. Proliferative retinopathy is the result of diabetes-induced damage sustained by the retinal capillary bed (choroid). Damage to the choroid elevates oxygen deprivation in the retina. The retinal tissue responds to its anoxic environment by producing angiogenic cytokines that stimulate neovascularization. Neovascularization of the retina causes bleeding in the eye, retinal scar tissue, retinal detachments, and any of one of these symptoms can cause decreased vision or blindness. Diabetics often also suffer from neovascular glaucoma, which manifests in rubeosis, blood vessels growing on the iris that causes closure of the angle.

Thus, diseases or conditions caused by any of the foregoing diabetic ocular diseases can be treated in accordance with the disclosed CO formulations and methods.

2. Choroidal Maladies

Examples of choroidal maladies amenable for treatment with the disclosed CO formulations and methods include, but are not limited to, choroidal neovascularization, polypoidal choroidal vasculopathy, central sirrus choroidopathy, a multi-focal choroidopathy or a choroidal dystrophy (e.g., central gyrate choroidal dystrophy, serpiginous choroidal dystrophy, or total central choroidal atrophy).

In some forms, the subject to be treated is suffering from polypoidal choroidal vasculopathy (PCV). PCV is an abnormal choroidal vasculopathy that is believed to be a variant of type 1 neovascularization, although it has been proposed that PCV is a distinct vascular abnormality of choroidal vessels (Imamura et al. (2010). Survey of Ophthalmology, volume 55, pp. 501-515). In some embodiments, a subject in need of treatment is enterally, for example orally, administered an effective amount of the CO formulation. In a further embodiment, the CO formulation is administered intravenously. Clinical manifestations of PCV include vascular abnormalities and variably sized serous, serosanguineous detachments of the neurosensory retina and pigment epithelium around the optic nerve or in the central macula. Subretinal exudation and/or bleeding can also be experienced by subjects with PCV. In another form, the PCV subject has lipid depositions in the eye. The disclosed formulations and methods may reduce the occurrence and/or severity of a PCV clinical manifestation experienced by the PCV subject treated with the methods described herein, compared to the occurrence and/or severity of the clinical manifestation prior to treatment. For example, a patient receiving treatment for PCV with one of the non-surgical treatment methods provided herein, experiences a reduction in the occurrence and/or severity of a vascular abnormality.

3. Macular Degeneration

In some forms, the subject to be treated suffers from macular degeneration. The macula is a region of the retina that contains an elevated concentration of the photo-sensor cells that are responsible for fine-detail vision. Macular degeneration is the name given to a poorly understood group of diseases that cause the photosensor cells of the macula to lose function. The result of macular degeneration is the loss of vital central vision and detailed vision. A subject stricken with macular degeneration experiences a blank spot in the center of their visual field and often loses the ability to read small print. (Source: Macular Degeneration Foundation, San Jose, Calif.: found at eyesight.org website)

Over 12 million Americans have some form of macular degeneration. One in six Americans between the ages of 55 and 64 will be affected by macular degeneration and the incidence of the disease increases with age. Each year 1.2 million of the estimated 12 million people with macular degeneration will suffer severe central vision loss. Each year 200,000 individuals will lose all central vision in one or both eyes.

Although the exact cause of macular degeneration is unknown, the architecture of the macula reveals clues as to how the disease might be initiated. The macula contains highly active photoreceptors that consume a great deal of energy. Generating this energy requires a rich supply of oxygen and nutrients. The macula has one of the highest rates of blood-flow through its supply-vessels (i.e., choroid). Anything that interferes with this rich blood supply can cause the macula to malfunction. The oxygen-deprived macula responds by producing cytokines that signal endothelial cell growth and neovascularization.

There are two basic types of macular degeneration: dry-form and wet-form. Approximately 85% to 90% of the cases of macular degeneration are the dry type. In the dry form of the disease, the deterioration of the retina is associated with the formation of yellow deposits under the macula known as drusen. The deposition of drusen correlates with decrease in the thickness of retinal cells that make up the macula. The amount of central vision loss is directly related to the location and severity of the drusen-induced retinal thinning. The dry-form of macular degeneration tends to progress more slowly than the wet-form of the disease. There is currently no effective treatment for dry-form macular degeneration. A small percent of individuals suffering from the dry-form of macular degeneration progress to the wet-form of macular degeneration.

The wet-form of macular degeneration is a rapidly progressing disease that almost always results in severe vision loss. Vision-loss associated with Wet macular degeneration is the result of sub-retinal neovascularization. The rapid growth of the sub-retinal blood vessels causes the overlying layer of retinal cells to buckle and become detached from the nutrient-rich choroid. In extreme cases of wet macular degeneration, the proliferating vessels penetrate the retina and infiltrate the vitreous humor. Several treatments exist for wet-form neovascularization, however, none are remotely satisfactory. Thus, symptoms caused by macular degeneration including degeneration of the photoreceptors and retinal pigment epithelium can be treated in accordance with the disclosed CO formulations and methods.

4. Traumatic Optic Neuropathy

The ocular disease can be traumatic optic neuropathy (TON). TON is a rare cause of vision loss which occurs following violent or penetrating head trauma, with severe effects, particularly when both optic nerves are involved (Yu-Wai-Man, Taiwan J Ophthalmol, 5(1): p. 3-8 (2015)). Young adult males in their early 30s account for 79-85% of the affected group. Motor vehicle/bicycle accidents (49%), falls (27%), and assaults (13%) are the most common causes of TON in this patient population (Levin, et al., Ophthalmology, 106(7): p. 1268-77 (1999), Lee, et al., Eye (Lond), 24(2): p. 240-50 (2010)). Most of the occurrences of TON in children occur as a result of falls (50%) and traffic accidents (40%) (Mahapatra & Tandon, Pediatr Neurosurg, 19(1): p. 34-9 (1993)). Vision loss, dyschromatopsia, visual field abnormalities, and a relative afferent pupil defect are some of the clinical symptoms of TON. There are two types of TON: indirect TON and direct TON. Indirect TON is caused by trauma to the head or face, which causes energy from the impact to be passed onto the bone structures that carry the optic nerve. Sheering forces act to weaken either the nerve or the pial vasculature. Direct TON refers to nerve damage caused by mechanical forces applied directly to the nerve, such as avulsion or laceration, or nerve impingement from a variety of sources, such as a penetrating foreign body, displaced fracture fragment, or optic canal fracture (Chaon & Lee, Curr Opin Ophthalmol, 2015. 26(6): p. 445-9 (2015)). Thus, optic nerve dysfunction leads to axon degeneration and subsequent death of retinal ganglion cells (RGCs), which transmit visual signals from the retina to the brain via the optic nerve, and even irreversible vision loss (Stutzki, et al., Front Cell Neurosci, 8: p. 38 (2014), Schmitt, et al., Mol Neurodegener, 9: p. 39 (2014)). Given the neuroprotective effect of oral CO treatment, TON can be treated using the CO formulations and procedures provided.

5. Retinopathy of Prematurity

The ocular disease can be retinopathy of prematurity. In the United States, 1 of every 10 infants is born prematurely and will undergo a number of severe complications affecting multiple organs/tissues. Many of these pathologies can result in death of the premature infant or in the occurrence of debilitating chronic conditions that are likely to impact the future development and well-being of the affected child. One such conditions is retinopathy of prematurity (ROP), characterized by abnormal growth of blood vessels in the incompletely vascularized retina of extremely low gestational age neonates. Retinal vessel overgrowth in ROP results from the toxic effects of oxygen therapy necessary to the premature child for survival during immediate post-natal life. Preventive strategies involve fine-tuned perinatal care protocols to optimize oxygen exposure and limit the occurrence of co-morbidities. However, within 3.9 million infants born in the U.S. each year about 14,000 are affected by ROP, among them, 400-600 will become legally blind and many more will suffer long-term ocular complications, such as severe myopia and glaucoma (Casteels, et al., Eur J Pediatr, 171(6): p. 887-93 (2012)). When the disease develops, Ophthalmologists are challenged by the potentially harmful side effects of presently available therapies, such as complete blockade of normal vascular growth or damage to the neuroretina. Epidemiological data derived from World Health Organization (WHO) and the National Eye Institute (NEI) show that ROP relative incidence varies in different regions of the World and depends on the level of perinatal care (Cavallaro, et al., Acta Ophthalmol, 92(1): p. 2-20 (2014), Shah, et al., World J Clin Pediatr, 5(1): p. 35-46 (2016), Hartnett, et al., Graefes Arch Clin Exp Ophthalmol, 231(8): p. 433-8 (1993)). Low birth weight (less than 1250 grams), low gestational age (less than 31 weeks), and prolonged oxygen therapy have been consistently related to disease onset (Cavallaro, et al., Acta Ophthalmol, 92(1): p. 2-20 (2014), Shah, et al., World J Clin Pediatr, 5(1): p. 35-46 (2016), Hartnett, et al., Graefes Arch Clin Exp Ophthalmol, 231(8): p. 433-8 (1993)). However, a number of comorbidities are also indicated as potential risk factors (Yu-Wai-Man, Taiwan J Ophthalmol, 5(1): p. 3-8 (2015), Levin, et al., Ophthalmology, 106(7): p. 1268-77 (1999), Lee, et al., Eye (Lond), 24(2): p. 240-50 (2010)). When treated, the affected children may still have vision loss, and even if the treatment works, babies with ROP are more likely than others to develop conditions or diseases of the eye later in life, such as nearsightedness, strabismus, amblyopia, and glaucoma (Hartnett, et al., Graefes Arch Clin Exp Ophthalmol, 231(8): p. 433-8 (1993), Choi, et al., Br J Ophthalmol, 84(2): p. 138-43 (2000), Knight-Nanan, et al., Br J Ophthalmol, 80(4): p. 343-5 (1996), Kaiser, et al., Ophthalmology, 108(9): p. 1647-53 (2001)). To date, therapies are applied in the most advanced stages of the disease and essentially involve ablation of the peripheral retina through surgical procedures. These include laser photocoagulation, cryotherapy, and vitrectomy (Falavarjani & Nguyen, Eye (Lond), 27(7): p. 787-94 (2013), Hartnett & Penn, N Engl J Med, 367(26): p. 2515-26 (2012), Mutlu & Sarici, Int J Ophthalmol, 6(2): p. 228-36 (2013)). Intravitreal injections of anti-VEGF (Vascular endothelial growth factor) are also used to halt retinal neovascularization (RNV) in the most advanced stages of (Falavarjani & Nguyen, Eye (Lond), 27(7): p. 787-94 (2013), Hartnett & Penn, N Engl J Med, 367(26): p. 2515-26 (2012), Mutlu & Sarici, Int J Ophthalmol, 6(2): p. 228-36 (2013)). Thus, considering the benefits of CO on preserving retinal cell loss, ROP can be treated in accordance with the disclosed CO formulations and methods.

6. Retinitis Pigmentosa

The ocular disease can be retinitis pigmentosa (RP). RP is a set of rare hereditary illnesses characterized by cell breakdown and loss in the retina, which is the light-sensitive tissue that lines the back of the eye. Typical symptoms include trouble seeing at night and a loss of peripheral (side) vision. RP is classified as an uncommon condition. Although current figures are unavailable, it is widely assumed that the disorder affects around one in every 4,000 persons in the United States and around the world. In the early stages of RP, rods are more severely impacted than cones. People who lose their rods endure night blindness and a progressive loss of their visual field, which is the area of space viewable at any given time without moving their eyes. The loss of rods finally results in a breakdown and loss of the cones. As cones die in the later stages of RP, affected individuals tend to lose more of their visual field, thereby acquiring tunnel vision. These affected individuals may struggle with basic daily tasks such as reading, driving, walking without assistance, and/or recognizing persons and objects. (Source: www.nei.nih.gov). Retinitis pigmentosa (RP) pathology is characterized by diffused progressive failure of primarily rod photoreceptors, followed by degeneration of cone photoreceptors and the retinal pigment epithelium (RPE). Thus, RP can be treated in accordance with the disclosed CO formulations and procedures, given the benefits of CO in preserving retinal cell loss.

7. Sickle Cell Retinopathy

The ocular disease can be sickle cell retinopathy. Sickle cell retinopathy is an ocular condition of sickle cell disease, a category of hereditary hemoglobinopathies with a variety of systemic and ocular manifestations. The incidence of sickle cell trait (AS) is approximately 8% in African-Americans in North America, while SCD is 0.4 percent. Sickle cell (SC), Hemoglobin C (AC), and S-Thalassemia (S-Thal) genotypes are present in 0.2 percent, 2%, and 0.03 percent of African-Americans in North America, respectively. Notably, the incidence of proliferative retinopathy is highest in individuals with SC or S-Thal (33% and 14%, respectively), while patients with Hemoglobin SS has a 3% incidence (Fekrat, Sickle Retinopathy. New York: Thieme Medical Publishers: New York (1998)). Normal blood cells, which are round and oval in shape, may readily travel through tiny blood channels such as capillaries. On the other hand, local hypoxic circumstances alter the form of red cells in SCD patients, resulting in rigid, sickle-shaped red blood cells (RBCs) due to the irreversible conversion of soluble hemoglobin into crystalline hemoglobin. Hypoxia-induced changes in the adhesion capabilities of capillary endothelial cells result in decreased vascular flow and channel blockage (Michiels, et al., Biochim Biophys Acta, 1497(1): p. 1-10 (2000)). The trapping of sickle-shaped red cells in small blood arteries, capillaries and veins in various parts of the eye, both anterior and posterior segments, causes distinctive damage. Clinical signs differ depending on the presence or absence of vaso-proliferative alterations. As a result, this illness is characterized as non-proliferative sickle cell retinopathy (NPSR) or proliferative sickle cell retinopathy (PSR) (Emerson & Lutty, Hematol Oncol Clin North Am, 19(5): p. 957-73, ix. (2005)). The pathophysiology of SCD is heavily influenced by oxidative stress (Vona, et al., Antioxidants (Basel), 10(2) (2021)). Given the positive effects of low-doses of CO on oxidative stress, SCR can be treated using the disclosed CO formulations and processes.

8. Effective Amounts

The effective amount can be a dosage sufficient to treat, inhibit, or alleviate one or more symptoms of an ocular disease, disorder or condition being treated or to otherwise provide a desired pharmacologic and/or physiologic effect. The effective amount of the CO formulations will vary from subject to subject, and can depend on the species, age, weight and general condition of the subject, the severity of the ocular disorder being treated, and its mode of administration. Effective dosages and schedules for administering the therapeutics may be determined empirically. The dosage ranges for the administration of the formulations are those large enough to effect one or more desired responses.

Exemplary dosages are disclosed herein including above and tested in the experiments below. For example, in some embodiments, the methods include administering a therapeutically effective dose of the liquid CO formulation to the subject. In some forms, the methods include providing a dose sufficient to achieve at least 2%, at least 3%, at least 4%, at least 5%, at least 6%, at least 7%, at least 8%, at least 9%, or at least 10%, up to about 12%, 13%, 14%, or 15% Carboxyhemoglobin (COHb) peak saturation of red blood cells (RBC). In some embodiments, the dose achieves 4% to 10% peak COHb saturation range.

9. Timing of Administration and Regimens

As introduced above, the timing and number of administrations, and dosage thereof can vary based on the subject, and the nature of the disease and its severity. Exemplary treatment regimens are illustrated in the experiments below. For example, in some embodiments, the methods include enterally, for example orally, administering a single dose. In some embodiments, the methods include orally administering a double or triple dose split into equal doses delivered at about 60 minutes apart. In some embodiments, the methods include enterally, for example orally, administering a single or doubly daily dose for a number of days or weeks. In some embodiments, the methods include orally administering a daily dose on a sub-chronic or chronic basis. In some embodiments, the methods include administering an alternate day, twice per week, weekly or bi-weekly dose on a sub-chronic or chronic basis. Thus, the dosage regimen can be a daily dosage, or any other dosage regimen consistent with the disclosed methods. The timing of the administration of the composition will also depend on the formulation and/or route of administration used. The compound may be administered once daily, but may also be administered two, three or four times daily, or every other day, or once or twice per week. For example, the subject can be administered one or more treatments 1, 2, 3, 4, 5, 6, 7, 8, 9, 10, 11, 12, 13, 14, 15, 16, 17, 18, 19, 20, 21, 22, 23, or 24 hours, days, weeks, or months apart. In some embodiments, the composition is administered according to the regimen for a specific period, e.g., days, weeks, or months, or years; in some embodiments, the composition is administered according to the regimen for an unspecified time; in some embodiments, the composition is administered for a combination of specified and unspecified periods.

10. Route of Administration

The methods typically included treating an ocular disorder by administering to the subject an effective amount of an enteral CO formulation. For example, a subject can be administered an effective amount of a pharmaceutical formulation containing dissolved carbon monoxide and a pharmaceutically acceptable carrier. The composition is typically administered enterally (e.g., via the gastrointestinal tract). Thus, the composition is typically administered via the gastrointestinal tract, including without limitation oral, topical, sublingual, gastric, or rectal administration. In some embodiments, the composition is administered by oral delivery. Other methods of administration are also contemplated and include, for example, injection, rectal inserts, gastric tube, gastro-duodenal tube, gastrostomy tube, gastrojejunostomy tube, cecostomy tube, rectal tube, sublingual, topical, etc. Topical administration can include application to the lungs, nasal, oral (sublingual, buccal), vaginal, or rectal mucosa.

It is to be understood that the disclosed method and formulations are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### EXAMPLES

### Example 1: Effect of a Liquid CO Formulation on Carboxyhemoglobin (COHb) Levels in the Blood

Methods

Male C57BL/6J mice (8-10 weeks old) were given either single or double dose of an aqueous liquid CO drug product orally. The single dose was administered at 2 mg carbon monoxide (CO) per kilogram (kg) or 10 mL/kg. The double dose was administered as a total dose of 4 mg CO/kg split into two equal doses of 2 mg CO/kg (10 mL/kg). Each of the two doses were administered 60 minutes apart. The mice were pretreated with a vehicle to establish the baseline levels of COHb in the blood. A sample size of 2-3 animals were used per group.

Results and Conclusion

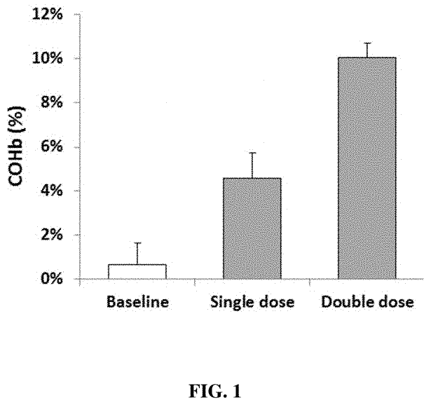

An aqueous liquid CO drug product herein referred to as “HBI-002” increased carboxyhemoglobin (COHb) levels in the blood to low, therapeutically acceptable levels in the mice. FIG. 1 is a bar graph showing the peak blood COHb levels in mice following either a single or double oral dose of HBI-002. As shown in FIG. 1, oral administration of HBI-002 provides appropriate blood levels of CO in the C57BL/6J mice in a dose-dependent manner. Single dosing at 2 mg/kg resulted in a 5% peak COHb, peaking at 10 minutes following drug administration (FIG. 1). Double dosing at 4 mg/kg split into two equal doses administered 60 minutes apart resulted in approximately 10% peak COHb in the blood, peaking at 5 to 20 minutes following administration of the second dose (FIG. 1). Similar observations were made when HBI-002 was administered to rats, dogs, and swine. These results indicate that oral administration of single or double doses of HBI-002 increases COHb in the blood to levels that are generally safe i.e., non-toxic.

### Example 2: Effect of HBI-002 on Retinal Neurovascular Injury in Retinal Ischemia-Reperfusion (I/R) in Mice

Diabetic Retinopathy (DR) is a neurovascular disease (Rudraraju, et al., Pharmacol Res, 161: p. 105115 (2020), Steinle, Mol Vis, 26: p. 355-358 (2020), Li, et al., Pharmacol Res, 159: p. 104924 (2020), Gardner & Davila, Graefes Arch Clin Exp Ophthalmol, 255(1): p. 1-6 (2017), Moran, et al., Am J Physiol Heart Circ Physiol, 311(3): p. H738-49 (2016)) and previous studies have reported that ischemia-induced oxidative stress and inflammation plays pathophysiological roles in development of the vascular pathology. The protection of neuronal cells from early ischemia/oxidative stress and inflammation offers a strategy to prevent the development of vascular lesions. CO using iCO or CORMs have been shown to induce HO-1 and protect retinal neurons against ischemic, oxidative and inflammatory insult (Ulbrich, et al., J Neuroinflammation, 14(1): p. 130 (2017), Ulbrich, et al., PLoS One, 11(10): p. e0165182 (2016), Ulbrich, et al., Graefes Arch Clin Exp Ophthalmol, 254(10): p. 1967-1976 (2016), Chen, et al., Biochem Biophys Res Commun, 469(4): p. 809-15 (2016)). Hence, the CO/HO-1 pathway represents a viable therapeutic target to ameliorate key events of ischemic retinopathies that are relevant to DR and other ischemic injury.

Methods:

Animal Model of Retinal Ischemia/Reperfusion (I/R) Injury

Ischemic injury plays a central role in Diabetic Retinopathy (DR). A mouse model of retinal ischemia/reperfusion (I/R) injury was used to evaluate the efficacy of HBI-002 administration in retinal diseases. I/R injury in groups of 8-12-week-old male and female C57BL/6J mice (n=11 per group; at least 3 males per group) was performed as described in previous studies (Fouda, et al., Cell Death Dis, 9(10): p. 1001 (2018), Shosha, et al., Cell Death Dis, 7(11): p. e2483 (2016)). Briefly, mice were anesthetized, and proparacaine (0.5%; Akorn) was applied to both eyes. Tropicamide (1%; Akorn) was used to dilate the pupil prior to surgery. A 32-gauge needle cannulated in the anterior chamber of the right eye was used to infuse sterile saline. Intraocular pressure was raised to 110 mmHg to create ischemia of the retina, by elevating the saline reservoir, and maintained for 40 minutes after which, reperfusion was accomplished by removal of the 32-gauge needle. The blanching of the retina was monitored to confirm the loss of blood flow. The left eye was treated by briefly inserting a 32G needle into the anterior chamber through the cornea to serve as a sham control.

Drug Administration of HBI-002

HBI-002 is an oral drug product composed of an aqueous formulation containing CO. The HBI-002 drug product may also contain FDA-defined Generally Recognized as Safe (GRAS) components. HBI-002 is manufactured using a controlled, reproducible process to achieve the targeted CO concentration (Belcher, et al., PLoS One, 13(10): p. e0205194 (2018)). The vehicle contained the same formulation without the CO (Prusky, et al., Invest Ophthalmol Vis Sci, 45(12): p. 4611-6 (2004)).

I/R (right eye) mice were orally administered a single dose of 2 mg/kg (Belcher, et al., PLoS One, 13(10): p. e0205194 (2018)) 1 hour before I/R, then dosed daily for 7 days with two doses 60 min apart (the same dose regimen shown to be effective in Example 1), followed by a single dose per day until the end of the experiment. For controls, sham (left eye) mice were subjected to the same treatment regimen but received the vehicle (Belcher, et al., PLoS One, 13(10): p. e0205194 (2018)) instead of the HBI-002. A bilateral I/R control group was also tested. The mice were sacrificed at pre-determined times following ischemia (6 hours, 24 hours, 7 days, 14 days, and 28 days), and eyes were extracted and processed for further analysis as described below.

Oxidative/Nitrative Stress

Oxidative/nitrative stress was evaluated by dihydroethidium (DHE) staining of retinal cryosections and dot blot analysis of 3-nitrotyrosine and 4-hydroxynonenal formation as described in previous studies (Gutsaeva, et al., Int J Mol Sci, 21(15) (2020), Abouhish, et al., Antioxidants (Basel), 9(7) (2020)). Briefly, retinal cryosections from different experimental groups (6 and 24 hours post-I/R) were stained with 2 μM DHE solution and images were taken using a Zeiss Axioplan-2 microscope (Carl Zeiss) equipped with the Axiovision program (version 4.7; Carl Zeiss). For the dot blot assays, the equivalent amounts of proteins prepared from mouse retinal lysates (6 and 24 hours post-I/R) were spotted on nitrocellulose membranes and probed with either anti-3-nitrotyrosine (3-NT; 1:1000; Cayman) or anti-4-hydroxynonenal (4-HNE; 1:1000; Abcam) antibodies. Immuno-positive spots were visualized using a chemiluminescence-based assay (Bio-Rad). β-actin was used as the loading control. Scanned images of blots was used to quantify protein expression using NIH ImageJ software.

Evaluation of Neurodegeneration

Neuronal degeneration will be assessed as previously described (Fouda, et al., Cell Death Dis, 9(10): p. 1001 (2018), Shosha, et al., Cell Death Dis, 7(11): p. e2483 (2016)) ^{by} using the neuronal cell marker NeuN to label surviving neurons in whole retinal flat mounts (7 days post-I/R). On day 7 following I/R or sham surgery, eye balls will be collected and preserved overnight at 4° C. in 4% paraformaldehyde (PFA). Retinas will be dissected, permeabilized, blocked, and then tagged overnight at 4° C. with anti-NeuN antibody. The retinas then treated for 1 hour with Alexa-488 anti-mouse IgG. The retinas will be imaged using a confocal microscope after flat mounting (LSM 510; Carl Zeiss, Thornwood, N.Y., USA). The NeuN-positive cells will be counted using ImageJ software after four pictures are captured in the midperiphery of each retina. The results will be provided as a proportion of NeuN-positive cells of the I/R eyes compared to the sham eyes.

Retinal Vasculature Isolation and Measurement of Acellular Capillaries

The trypsin digestion method was used to isolate the retinal vasculature (7 days post-I/R) as previously described with minor modifications (Fouda, et al., Cell Death Dis, 9(10): p. 1001 (2018), Shosha, et al., Cell Death Dis, 7(11): p. e2483 (2016), Promsote, et al., Antioxid Redox Signal, 25(17): p. 921-935 (2016)). Briefly, eyeballs were removed and fixed with 4% paraformaldehyde (PFA) overnight. Retinas were carefully dissected and digested with 3% trypsin (in 0.1 M Tris buffer at pH 7.8) for 1.5 hours at 37° C. The isolated retinal vasculature was air-dried on silane-coated slides and stained with periodic acid—Schiff, and hematoxylin. Acellular capillaries were counted in 10 random areas of the mid-retina using ImageJ software.

Histology and Morphometric Analysis

Retinal morphology was assessed at 7-, 14- and 28-days post-I/R injury on H&E-stained plastic retinal sections using a Zeiss Axioplan-2 microscope. Briefly, mice were sacrificed on day 7 post-I/R, and eyes were processed for further analysis. Mouse eyes were enucleated, embedded in optimal cutting temperature mounting medium (Tissue-Tek), frozen on dry ice, and sliced to obtain cryostat sections (10 μm). Retinal morphology was assessed by performing hematoxylin and eosin (H&E) staining on frozen sections.

Morphometric analyses were performed to measure total retinal thickness. Additionally, retinal sections were immunostained using an anti-RNA binding protein with a multiple splicing (RBPMS) antibody to localize retinal ganglion cells. The number of ganglia RBPMS-positive cells was quantified. Measurements were made in three adjacent fields (peripheral, midperipheral, central) on the temporal and nasal side of the optic nerve at 200- to 300-μm intervals, resulting in a total of six measurements obtained per eye; the initial measurement was made approximately ˜200 μm from the optic nerve. Average retinal thickness and number of ganglion cells were presented as a percentage compared with the contralateral sham-operated eye. Proteins were extracted from retinas of sham-operated and I/R injured eyes as described in previous studies (Gardner & Davila, Graefes Arch Clin Exp Ophthalmol, 255(1): p. 1-6 (2017)) and subjected to SDS-PAGE and western blot using an anti-HO-1 antibody (1:1000). Scanned images of blots were used to quantify protein expression using NIH ImageJ software (rsb.info.nih.gov/ij/).

Immunofluorescence Staining

Glial cell activation was evaluated by anti-GFAP (Dako Cat. #Z0334; 1:200) immunofluorescence in retinal cryosections. Images were taken using a confocal microscope. The eye balls were snap frozen in optimal cutting temperature (OCT) solution. 10 μm thick cryostat sections were obtained, mounted on glass slides, permeabilized with 1% Triton for 10 minutes and blocked in 10% normal goat serum for 1 hour. Sections were then incubated in anti-GFAP primary antibody at 4° C. overnight. On the next day, sections were incubated for 1 hour at room temperature in fluorescein-conjugated secondary antibody, washed in PBS, and covered with mounting medium and DAPI. The retinal sections were imaged using a confocal microscope (LSM 510; Carl Zeiss, Thornwood, N.Y., USA).

Electroretinogram

Mice from different groups (28 days post-I/R), dark-adapted overnight, were anesthetized and proparacaine (0.5%) drops were applied to both eyes and pupils dilated with 1% tropicamide and 2.5% phenylephrine hydrochloride. Animals were placed on a heating pad controlled by a rectal thermometer and DTL (Dawson, Trick and Litzkow, US) electrodes were placed on the corneas and needle electrodes in the cheeks (references) and tail (ground). All experiments utilized a series of full-field light flashes presented in a Ganzfeld (LKC Tech). Flashes were presented from dim to bright with the interstimulus interval increasing with brightness.

After dark-adapted testing, animals were light-adapted for 10 minutes with a background light in the Ganzfeld (30 cd/m2). To record cone-isolated responses, a series of full-field flashes with increasing intensity were presented in the presence of the background light. Stimuli were generated by a custom LED-based system. A 5500° white LED was used for bright stimuli and a 470-nm blue LED for dim stimuli. The light from the blue LED passed through neutral density filters and defocused before collection by the optical fiber launcher to further diminish the light intensity. LED flashes of 5-millisecond (ms) duration was used (Promsote, et al., Antioxid Redox Signal, 25(17): p. 921-935 (2016)).

Optokinetic Tracking for Visual Acuity

Optomotor reflex-based spatial frequency thresholds were analyzed (14- and 28-days post-I/R) using a visuomotor behavior measuring system (OptoMotry, Cerebral Mechanics) as described in previous studies (Prusky, et al., Invest Ophthalmol Vis Sci, 45(12): p. 4611-6 (2004)). Briefly, all animals were habituated before the outset of testing with handling and by placing them on the platform for a few minutes at a time. The mice were tested during the first few hours of their daylight cycle. Tracking was defined as a reproducible smooth pursuit with a velocity and direction concordant with the stimulus. Spatial frequency threshold, a measure of visual acuity, was determined automatically with the optokinetic tracking software, which used a staircase paradigm based upon head-tracking movements. Rotation speed (12°/s) and contrast (100%) were kept constant. Mice were sacrificed on day 7 post-I/R, and eyes were processed for further analysis.

Statistical Analysis

Power analyses were performed to determine the number of animals required per group. All measurements were considered continuous variables and transformed as needed (e.g. log transformation to equalize standard deviations and produce normal distributions). Student t-tests were used for pairwise comparisons. Analysis of variance (ANOVAs) were used to test the overall significance of >2 groups. Pearson correlation was used to assess the association between two different measurements. Non-parametric Kruskal-Wallis or Mann-Whitney tests with Bonferroni correction for multiple comparisons was used for data that are not normally distributed. Statistical significance was set at p<0.05.

Results:

The efficacy of dual dosing in acute I/R injury in mice was evaluated to assess a higher peak COHb.

The Effect of HBI-002 Treatment on the Optokinetic Tracking Response in a Mouse Model of Retinal I/R Injury

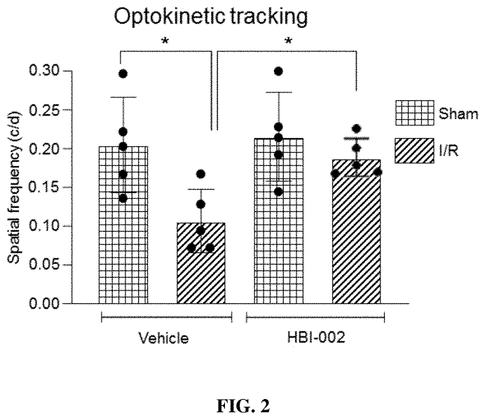

FIG. 2 is a bar graph illustrating the effect of HBI-002 treatment on the optokinetic tracking response in a mouse model of retinal I/R injury. The optokinetic tracking response was measured in sham-operated and IR-injured mice treated with vehicle or HBI-002 7 days following ischemic insult using the OptoMotry system. As shown in FIG. 2, I/R caused a marked decrease in visual function in vehicle treated mice as demonstrated by a reduction in spatial frequency threshold when compared to the sham eye from vehicle treated mice.

Treatment with HBI-002 significantly recovered visual function as evidenced by the increase in spatial frequency threshold in IR-injured eyes versus sham in HBI-002 treated mice (FIG. 2).

The Effect of HBI-002 Treatment on Histopathology in a Mouse Model of I/R Injury

Retinal I/R injury leads to neuronal cell loss, which is accompanied by morphologic distortions and thinning of the Retinal cell layer as described in previous studies. Similar observations were reported in previous studies (Gutsaeva, et al., Int J Mol Sci, 21(15) (2020), Palmhof, et al., Front Cell Neurosci, 13: p. 174 (2019), Yokota, et al., Invest Ophthalmol Vis Sci, 52(11): p. 8123-31 (2011)). The microscopic images showed hematoxylin and eosin stained retinal cryosections (mid-peripheral field) from IR-injured and sham-operated eyes of vehicle or HBI-002 treated mice 7 days after ischemic insult. Compared to sham animals treated with the vehicle, I/R animals treated with the vehicle demonstrated distorted morphologies with significant convolutions of the inner and outer nuclear layer (INL and ONL respectively). HBI-002-treatment in the I/R animals significantly ameliorated these distortions of the INL and ONL respectively. The lack of observations of RPE layer is likely due to the absence of RPE pathology in the I/R model in general. However, the other factors, such as oxidative stress and inflammation are key mediators of RPE injury in AMD and were observed in the RPE layer. Based on the effects of CO on antioxidant enzyme, HO-1, this formulation may be beneficial in protecting RPE layer in an AMD pathology/mouse model.

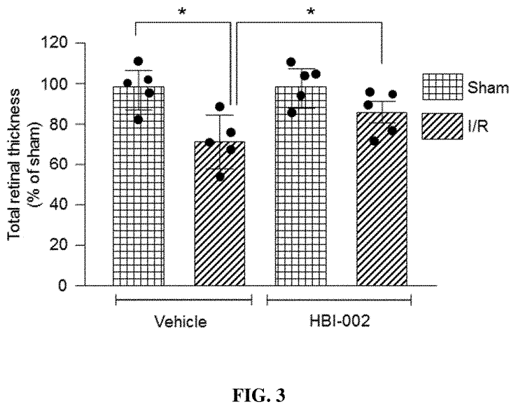

FIG. 3 is a bar graph of the morphometric analysis of retinal cryosections to measure total retinal thickness. As shown in FIG. 3, H&E staining revealed that I/R resulted in a significant reduction in total retinal thickness in vehicle treated mice as compared with HBI-002 treated mice. Preservation of both the total retinal thickness and the inner nuclear layer (INL) thickness was observed in IR-injured eyes of HBI-002 treated mice (FIG. 3).

The Effect of HBI-002 Treatment on the Induction of Ganglion Cell Loss in Retinal I/R-Injured Mice

Since the degeneration of neuronal cells in the ganglion cell layer is an important characteristic of the retinal damage following I/R and important in DR (Yang, et al., Med Res Rev., 40(4):1147-1177 (2019), Ryter, Arch Biochem Biophys, 678: Article 108186. PMID: 31704095 (2019)), whether HBI-002 treatment could ameliorate the loss of retinal ganglion cells was evaluated.

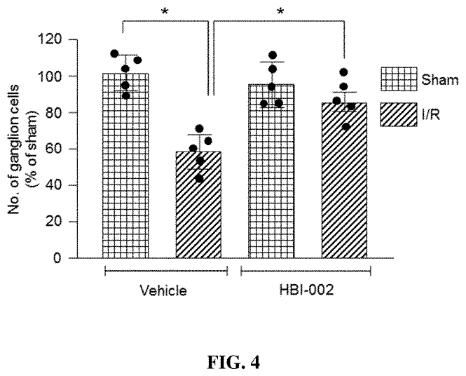

In confocal micrographs not shown illustrate representative immunofluorescence images of retinal cryosections from IR-injured and sham-operated eyes of vehicle or HBI-002 treated mice 7 days following ischemic insult identifying retinal ganglion cells. Green fluorescence staining of RBPMS-positive cells indicated ganglion cells. Nuclei were stained with 4′,6-diamidino-2-phenylindole (DAPI; blue). FIG. 4 is a bar graph quantifying the RBPMS-positive cells. The data are expressed as cell number/100 μm retinal length and presented as a percent change from sham animals. Consistent with H&E data, analysis of the number of ganglion cells, identified as cells immune-positive to RBPMS, in vehicle and HBI-002 treated mice revealed that HBI-002 treatment significantly reduced retinal ganglion cell loss (FIG. 4).

FIG. 5 illustrates the changes in Heme Oxygenase-1 (HO-1) expression in the retina of vehicle and HBI-002 treated I/R mice. Shown in FIG. 5 are representative immunoblotting images of retinal cryosections from IR-injured and sham-operated eyes of vehicle or HBI-treated mice 7 days after ischemic insult to measure HO-1 protein levels. Also shown in FIG. 5 is a bar graph quantifying HO-1 expression. The results were normalized to β-actin levels and expressed as fold change. HBI-002 treatment induced the upregulation of HO-1 protein expression in the retinas of vehicle and HBI-002 treated mice (FIG. 5). This result indicates that CO increases protective gene expression as a mechanism of action.

Conclusions

These provided results support HBI-002 as protective for retinal health and function. Generally, the data indicate significant improvement in retinal histopathology, ganglion cell loss, and optokinetic tracking with HBI-002 treatment and potentially a significant improvement in both neuronal and vascular outcomes resulting in a mouse model of retinal I/R injury. Although dual dosing was used in these experiments, results support a conclusion that single daily dosing of HBI-002 would also be effective to provide significant improvement in I/R related changes.

### Example 3: Effect of HBI-002 on Retinal Neuronal Injury and Visual Function in Diabetic Mice

Most prior studies of retinal CO therapy have been acute, and therefore long-term benefits of CO therapy have not been evaluated. Further, no study of CO therapy has been conducted under diabetic retinopathy (DR) conditions. The Heme oxygenase isoform, HO-1 mRNA is upregulated in the retinas of diabetic rats; however, the level of HO-1 induction under stress conditions is not sufficient to combat oxidative stress and inflammation (Cukiernik, et al., Curr Eye Res, 27(5):301-8 (2003)). Pharmacological induction of HO-1 can overcome this hurdle and provide substantial protection (Fa, et al., Invest Ophthalmol Vis Sci., 53(10): 6541-56. PMID: 22661484 (2012)). Even though these prior studies attest to the fact that HO-1 induction/CO therapy is a viable therapeutic strategy, it has not been advanced toward clinical use. These studies using Streptozotocin (STZ) diabetic mice confirms the findings of retinal I/R injury models, and provide details on the long-term usage of HBI-002 in the treatment of DR.

Methods:

Induction of Experimental Diabetes in Mice

To test the relevance of HBI-002 therapy on long-term retinal changes associated with diabetes, diabetic mice using STZ were created per standard protocol as previously reported (Martin, et al., Invest Ophthalmol Vis Sci, 45(9): p. 3330-6 (2004), Gambhir, et al., Invest Ophthalmol Vis Sci, 53(4): p. 2208-17 (2012), Martin, et al., Mol Vis, 15: p. 362-72 (2009)). Briefly, groups of six-week-old male C57BL/6J mice received an intraperitoneal injection of 75 mg/kg STZ dissolved in sodium citrate buffer (0.01 M, pH 4.5) on three consecutive days. One week later, mice with fasting blood glucose levels higher than 250 mg/dL were considered to be diabetic (DB). Age-matched, non-diabetic C57BL/6J mice were used as controls. Different sets of control and DB mice were dosed daily with HBI-002 or vehicle (2 mg/kg or 10 ml/kg 5×/week, p.o.) starting 2 weeks after diabetes was confirmed (blood glucose >250 mg/dl). Mice were sacrificed at the end of 8, 20, and 24 weeks and various structural and functional changes associated with DR (Sergeys, et al., Invest Ophthalmol Vis Sci., 60(2): 807-822 (2019)), were measured as described below.

Measurement of Oxidative/Nitrative Stress

Oxidative/nitrative stress was evaluated by dihydroethidium (DHE) and 4-hydroxynonenal staining of retinal cryosections and dot blot analysis of 3-nitrotyrosine and 4-hydroxynonenal formation as previously reported (Gutsaeva, et al., Int J Mol Sci, 21(15) (2020), Abouhish, et al., Antioxidants (Basel), 9(7) (2020)). Briefly, retinal cryosections from different experimental groups were stained with 2 μM DHE solution, and the images were taken using a Zeiss Axioplan-2 microscope (Carl Zeiss) equipped with the Axiovision program (version 4.7; Carl Zeiss). For dot blot assays, equivalent amounts of proteins prepared from mouse retinal lysates, were spotted on nitrocellulose membranes, and probed with either anti-3-nitrotyrosine (3-NT; 1:1000; Cayman) or anti-4-hydroxynonenal (4-HNE; 1:1000; Abcam) antibodies. Immuno-positive spots were visualized using a chemiluminescence-based assay (Bio-Rad). β-actin was used as the loading control.

Results:

HBI-002 Treatment Reduces Retinal Oxidative Stress in 8 Weeks of Diabetic Mice.

Oxidative/nitrative stress was evaluated by dihydroethidium (DHE) and 4-hydroxynonenal staining of retinal cryosections and dot blot analysis of 3-nitrotyrosine and 4-hydroxynonenal formation. 8 weeks of sustained hyperglycemia significantly elevated oxidative/nitrative stress in the retina of DB mice. However, HBI-002 treatment (2 mg/kg or 10 ml/kg 5×/week) significantly improved these early deleterious changes associated with the diabetic phenotype (Data not shown).

HBI-002 Treatment Improves Body Weight, Blood Glucose, and Visual Acuity in 20 Weeks of Diabetic Mice.

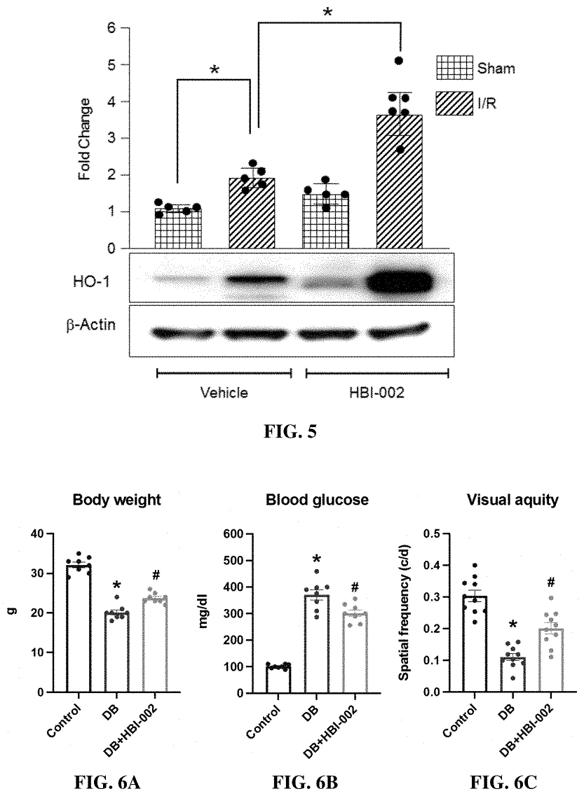

Chronic hyperglycemic condition in mice is associated with decreased body weight and hyperglycemia. Therefore, changes in these parameters were evaluated following HBI-002 treatment in 20 weeks of diabetic mice. As shown in FIGS. 6A and 6B respectively, 20-weeks post-DB significantly decreased body weight and induced severe hyperglycemia in mice. Interestingly, the HBI-002 treatment marginally improved body weight and blood glucose levels in DB mice (FIGS. 6A and 6B).

Additionally, loss of visual acuity is a long-term pathological feature associated with chronic hyperglycemia. Therefore, optokinetic tracking for visual acuity was evaluated in all the experimental groups 20 weeks post-DB. Furthermore, Optomotor reflex-based spatial frequency thresholds were analyzed 20 weeks post-diabetes in mice using a visuomotor behavior measuring system (OptoMotry, Cerebral Mechanics) as previously described (Prusky, et al., Invest Ophthalmol Vis Sci, 45(12): p. 4611-6 (2004)). In accordance with other reports, a significant loss of visual acuity in 20 weeks DB mice was observed (FIG. 6C). However, HBI-002 treatment (2 mg/kg or 10 ml/kg 5×/week) improved visual acuity in diabetic mice (FIG. 6C).

### Example 4: HBI-002 Treatment Improves Vascular Leakage in the Retina of 20-24 Weeks Diabetic Mice

Increased vascular permeability is a feature of diabetic retinopathy. The vasculature of the retina, which ordinarily has tight control over the fluid and blood components that enter the retina, becomes leaky in diabetes, resulting in increased albumin flux into the retina, fluid buildup, and macular edema, and may develop into bleeding vessels over time. Changes in vascular permeability were evaluated in 20-24 weeks DB mice using two techniques, 1) Florescence angiography and 2) Evans blue dye leakage analysis.

Methods:

Fluorescence Angiography

Mice (20-24 weeks post-DB) were anesthetized, and their eyes dilated using a 1% tropicamide eye drop. Each mouse was placed on the imaging platform of the Phoenix Micron III retinal imaging microscope (Phoenix Research Labs). To keep the eye moist during imaging, lubricant gel was applied. For fluorescein angiography imaging of the retinal vasculature, 20 μL 10% fluorescein sodium (Apollo Ophthalmics) was injected into the mice intraperitoneally, followed by rapid acquisition of fluorescent images for −5 minutes. Fluorescein leakage was assessed as indistinct vascular borders progressing to diffusely hazy fluorescence.

Evans Blue Extravasation

To complement FA analysis, Evans blue extravasation was also assessed in mice as an additional parameter for vascular leakage analyses. To assess retinal vascular barrier function, Evans blue dye (EB) (Sigma-Aldrich, St. Louis, Mo., USA) was used as described previously (Luo et al., Investigative Ophthalmology & Visual Science, 52(10): p. 7556-7564 (2011)). Mice were deeply anesthetized, and EB dye, dissolved in normal saline (30 mg/mL), was injected intraperitoneally. The dye was allowed to circulate through the body for 2 hours. For morphological studies, the eyes were enucleated and fixed in 4% paraformaldehyde at room temperature for 1 hour, and then the retinas were excised, flat-mounted, examined, and imaged with a Zeiss Axioplan 2 fluorescence microscope (Carl Zeiss, Thornwood, N.Y., USA).

Results:

Changes in fluorescence angiography in vehicle or HBI-002-treated DB mice post-20 weeks of diabetes induction were observed (data not shown). The white arrows show leakage of major vessels, and white asterisks show leakage in minor vessels as indicated by diffused hazy fluorescence. Vehicle-treated DB mice showed apparent leakage in major and minor vessels. However, the appearance of vascular leakage in HBI-002-treated DB mice was characterized by a lesser appearance of minor vascular leakage and no major vessel leakage.

Next, Evans blue extravasation assay performed in postmortem retinal flat mounts complemented fluorescence angiography data. The vascular leakage was characterized by the leakage of dye outside vessels (data not shown). In accordance with fluorescence angiography data, HBI-002-treated DB mice significantly preserved vasculature and reduced vessel leakage. These observations indicate the vasculo-protective effects of HBI-002 in the retina of DB mice.

### Example 5: HBI-002 Treatment Improves 20 Weeks of Diabetes-Induced Retinal Thinning in Mice

Progressive retinal thinning due to cell loss in multiple retina layers is a hallmark feature of chronic hyperglycemia in humans and diabetic mice. Therefore, changes in overall and cell-layer-specific retinal thinning were evaluated in the vehicle, and HBI-002 treated DB mice using two analyses, 1) Hematoxylin and eosin (H&E) staining in retinal sections and 2) Optical coherence tomography (OCT) in anesthetized mice.

Methods:

Histology and Morphometric Analysis

Retinal morphology was assessed at 20 weeks post-confirmed diabetes on H&E-stained plastic retinal sections using a Zeiss Axioplan-2 microscope. Eyes were fixed in OCT (optimal cutting temperature) solution and cryopreserved. Serial sections (5 μm thickness) were prepared and stained with hematoxylin and eosin (H&E) such that morphologic analyses of central, temporal, and nasal regions of the retina (relative to the optic nerve) could be performed.

Optical Coherence Tomography (OCT)

To measure progressive retinal thinning in a DB mouse, OCT analysis was performed in vivo using a Bioptigen Spectral Domain Ophthalmic Imaging System (SDOIS; Bioptigen Envisu R2200) in mice at 20 weeks post-DB. The imaging protocol includes averaged single B scan, and volume intensity scans with images centered on the optic nerve head. In addition, the post-imaging analysis (a feature of the InVivoVue Diver 2.4 software [Bioptigen]) was carried out to measure the thickness of different retinal cell layers and the total retina (Promsote, et al., Antioxid Redox Signal, 25(17): p. 921-935 (2016)).

Results

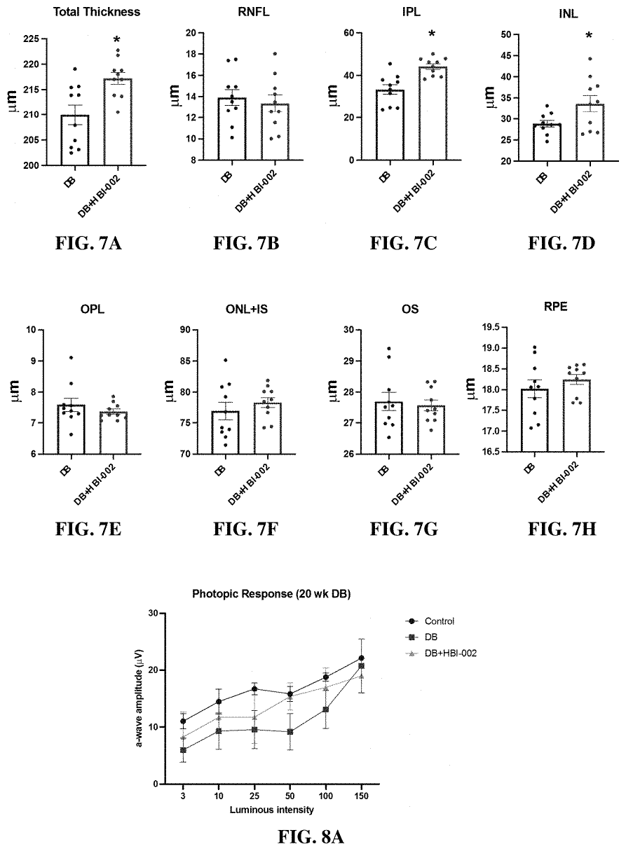

Vehicle-treated DB mice showed apparent retinal thinning due to 20 weeks of sustained hyperglycemia (data not shown). However, HBI-002-treated DB mice showed significant perseverance of retinal architecture. Next, OCT analysis was performed in anesthetized mice, and the thickness of the total retina and various retinal layers were calculated. SD-OCT tests showed well-organized retinal layers without any signs of disturbance to the inner or outer retina. HBI-002 treatment DB mice (20 weeks post-DB) demonstrated improved total retinal thickness (FIG. 7A), inner plexiform layer (IPL) (FIG. 7C), and inner nuclear layer (INL) (FIG. 7D). This analysis showed that HBI-002 treatment significantly blunted chronic hyperglycemia-induced retinal thinning in DB mice.

### Example 6: HBI-002 Treatment Improves Reactive Gliosis in 24 Weeks of Diabetic Mice

Hyperglycemia causes Müller cells to overexpress GFAP in the diabetic retina, resulting in reactive gliosis. Furthermore, persistent gliosis accelerates the neurodegenerative process during chronic hyperglycemia, resulting in direct and indirect retinal neuron loss (Bahr et al., Exp Eye Res., 186:107742 (2019)). Reactive gliosis was evaluated in non-diabetic (control) and DB mice treated with vehicle or HBI-002 20 weeks post-induction of diabetes.

Methods

Glial cell activation was evaluated by anti-GFAP (Dako Cat. #Z0334; 1: 200) immunofluorescence staining in retinal cryosections as described earlier (Thounaojam et al., Diabetes, 68(5):1014-1025 (2019), Thounaojam et al., J Clin Med., 9(6):1921 (2020)).

Retinal sections were fixed in 4% paraformaldehyde and incubated overnight at 4° C. with anti-mouse glial fibrillary acidic protein (GFAP) (1:200; Cayman Chemical, Ann Arbor, Mich., USA). Slides were washed three times with 0.1% Triton X-100 in 0.1 M phosphate-buffered saline (PBS) (pH 7.4), followed by incubation with appropriate fluorescence-conjugated secondary antibodies (Life Technologies, Eugene, Oreg., USA). Sections were mounted using a fluoroshield mounting medium with DAPI (4′,6-diamidino-2-phenylindole; Sigma-Aldrich, St. Louis, Mo., USA), and images captured at 20× magnification using Zeiss Axioplan-2 imaging fluorescence microscope (Carl Zeiss, Thornwood, N.Y., USA).

Results

Chronic hyperglycemia (20 weeks) was associated with severe reactive gliosis as indicated by projecting muller cell processing spanning throughout the retina (data not shown). Conversely, the HBI-002 treatment significantly reduced reactive gliosis, as indicated by reduced fluorescence projections in the retinal sections. This data agrees with the above-described observations that highlight the protective effects of HBI-002 treatment in improving chronic hyperglycemia-associated pathological changes in the mouse retina.

### Example 7: HBI-002 Treatment Improves Electroretinogram Response in 20 Weeks of Diabetic Mice

Alterations in Electroretinogram (ERG) responses are a prominent component of DR. Previous research has demonstrated that retinal function is substantially impaired in DR patients and experimental animals (Jackson et al., Vis Neurosci., 29(6):267-74 (2012), Samuels, et al., Vis Neurosci., 29(6):267-74 (2012)).

Therefore, ERG analysis was performed to understand the effects of HBI-002 treatment in improving retinal function in 20 weeks DB mice.

Methods

Electroretinogram (ERG)

Mice from different groups (28 days post-I/R), dark-adapted overnight, were anesthetized, and proparacaine (0.5%) drops were applied to both eyes and pupils dilated with 1% tropicamide and 2.5% phenylephrine hydrochloride. Animals were placed on a heating pad controlled by a rectal thermometer, and DTL electrodes were placed on the corneas and needle electrodes in the cheeks (references) and tail (ground). All experiments will use a series of full-field light flashes presented in a Ganzfeld (LKC Tech). Flashes will be presented from dim to bright, with the interstimulus interval increasing with brightness. After dark-adapted testing, animals will be light adapted for 10 minutes with a background light in the Ganzfeld (30 cd/m2). To record cone-isolated responses, a series of full-field flashes with increasing intensity were recorded in the presence of the background light. A custom LED-based system generated stimuli. A 5500° white LED was used for bright stimuli, and a 470-nm blue LED for dim stimuli. The light from the blue LED was intended to pass through neutral density filters and defocused before collection by the optical fiber launcher to diminish the light intensity further. LED flashes of 5-ms duration (Promsote, et al., Antioxid Redox Signal, 25(17): p. 921-935 (2016)).

Results

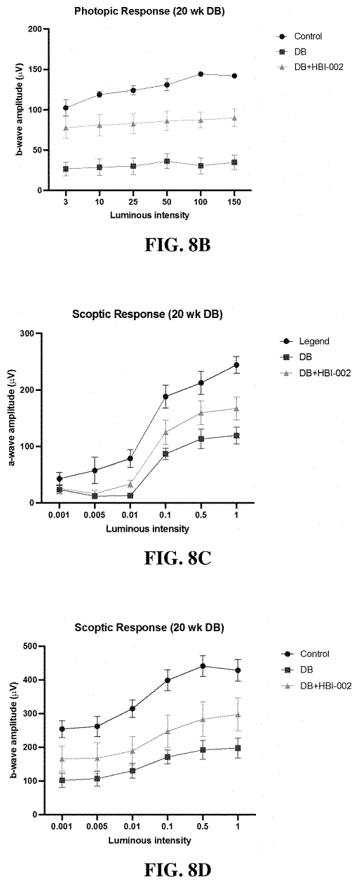

A- and b-wave responses in photopic (light) and scotopic (dark) conditions represent the firing of respective populations of retinal neurons in the visual pathway. Average scotopic a-wave and b-wave amplitudes are shown (FIGS. 8A-8D) for non-diabetic and DB mice treated with vehicle or HBI-002 20 weeks post-diabetes induction. The results demonstrate functional deficits in the diabetic retina 20 weeks post-diabetes. In addition, the ERG analysis confirmed the neuroprotective effect of HBI-002 treatment in diabetes-induced retinal dysfunction.

Those skilled in the art will recognize, or be able to ascertain using no more than routine experimentation, many equivalents to the specific embodiments of the method and compositions described herein. Such equivalents are intended to be encompassed by the following claims.

1. A method for treating or preventing an ocular disease, the method comprising administering to a subject in need thereof an enteral formulation comprising an effective amount of dissolved gaseous, or entrapped gaseous bubbles of, carbon monoxide (CO).

2. The method of claim 1, wherein the ocular disease is selected from the group comprising choroidal neovascularization, retinal neovascularization, ocular tumors, diabetic retinopathy, retinopathy of prematurity, retinoblastoma, neovascularization of the cornea, glaucoma, macular degeneration, ischemic retinopathy, optic neuropathy, diabetic macular edema (DME), uveitis, senile cataract, ischemic retinopathy, optic neuropathy, sickle cell retinopathy, retinitis pigmentosa, and traumatic optic neuropathy.

3. The method of claim 1, wherein the carbon monoxide is dissolved in a carrier.

4. The method of claim 3, wherein the carrier is a liquid.

5. The method of claim 4, wherein the liquid comprises water, ethanol, or both.

6. The method of claim 5, wherein the liquid comprises water.

7. The method of claim 5, wherein the formulation comprises one or more of proteins, polypeptides, peptides polysaccharides lipids, fats, oils; and alcohols having two or more carbon atoms.

8. The method of claim 7, wherein the formulation comprises lipid, oil, and/or fat.

9. The method of claim 1, comprising providing a dose of CO sufficient to achieve about 3% to about 20%, optionally 4% to 10% of carboxyhemoglobin (COHb) in the blood.

10. The method of claim 1, wherein the formulation comprises a concentration of CO of between about 100 mg/l and 4,400 mg/l, or between about 100 mg/l and 800 mg/l.

11. The method of claim 1 comprising administering the subject
(i) an acute dosing regimen comprising one day of dosing of the formulation;
(ii) a dosing regimen comprising daily, alternate daily, bi-weekly or weekly dosing of the formulation for total time period of less than a year
(iii) a chronic dosing regimen comprising daily, alternate daily, bi-weekly, or weekly doses of the formulation for an unspecified period of time, optionally 1 year or greater.

12. The method of claim 1, wherein the formulation is administered by oral, sublingual, gastric, rectal, nasal, or vaginal administration.

13. The method of claim 1, wherein the formulation is administered by injection, rectal inserts, gastric tube, gastro-duodenal tube, gastrostomy tube, gastrojejunostomy tube, cecostomy tube, or rectal tube.

14. The method of claim 1, wherein the formulation is administered orally.

15. The method of claim 1, wherein the formulation is an aqueous formulation.

16. The method of claim 1, wherein the subject has diabetes.

17. A method for treating or preventing diabetic retinopathy, the method comprising administering to a subject with diabetes an enteral formulation comprising an effective amount of dissolved gaseous, or entrapped gaseous bubbles of, carbon monoxide (CO).

18. The method of claim 17, wherein the formulation comprises a water-based carrier in which the CO is dissolved or entrapped, and one or more of a lipid, oil, and/or fat.

19. The method of claim 18, comprising providing a dose of CO sufficient to achieve about 3% to about 20%, optionally 4% to 10% of carboxyhemoglobin (COHb) in the blood.

20. The method of claim 19, wherein the formulation comprises a concentration of CO of between about 100 mg/l and 4,400 mg/l, or between about 100 mg/l and 800 mg/l.
